(19) United States
(12) Patent Application Publication
(10) Pub. No.: US 2013/0023643 A1
(43) Pub. Date: Jan. 24, 2013

(54) NUCLEAR LOCALIZATION SIGNAL PEPTIDES DERIVED FROM VP2 PROTEIN OF CHICKEN ANEMIA VIRUS AND USES OF SAID PEPTIDES

(71) Applicants: Meng-Shiou Lee, Taichung (TW); Jai-Hong Cheng, Taichung (TW); Yi-Yang Lien, Taichung (TW)

(72) Inventors: Meng-Shiou Lee, Taichung (TW); Jai-Hong Cheng, Taichung (TW); Yi-Yang Lien, Taichung (TW)

(73) Assignee: China Medical University, Taichung City (TW)

(21) Appl. No.: 13/554,911

(22) Filed: Jul. 20, 2012

(30) Foreign Application Priority Data
Jul. 21, 2011 (TW) 100125831
Feb. 20, 2012 (TW) 101105459

(51) Int. Cl. C07K 14/01; C07K 9/00; C12N 15/62; C12N 15/63; C07K 7/06; C07K 19/00

(52) U.S. Cl. 530/322; 530/350; 530/329; 530/395; 536/23.4; 435/320.1

(57) ABSTRACT

Disclosed herein are isolated peptides having nuclear localization activity and derived from the VP2 protein of chicken anemia virus (CAV). The isolated peptides are proven to be useful and effective in the nuclear delivery of a selected target substance, such as proteins, peptides, nucleic acids, pharmaceutically active agents, chemical substances, etc. The isolated peptide can transport a target substance, in particular a protein, into the nucleus of a mammalian cell by forming a conjugate with the target substance, or via an expression cassette capable of expressing a fusion protein having the isolated peptide and the target protein.

### CROSS-REFERENCE TO RELATED APPLICATIONS

This application claims priority of Taiwanese Application Nos. 100125831 and 101105459, filed on Jul. 21, 2011 and Feb. 20, 2012, respectively, in which Taiwanese Application No. 101105459 claims internal priority of Taiwanese Application No. 100125831.

### BACKGROUND OF THE INVENTION

1. Field of the Invention

This invention primarily relates to nuclear localization signal (NLS) peptides derived from the VP2 protein of chicken anemia virus (CAV). Said NLS peptides are useful and effective in the nuclear delivery of a selected target substance, such as proteins, peptides, nucleic acids, pharmaceutically active agents, chemical substances, etc., and therefore are expected to have potential for use in the fields of pharmacy, medicine, biotechnology, genetic engineering, etc.

2. Description of the Related Art

Nucleocytoplasmic transport, which is a process in eukaryotic cells that transports macromolecules, such as nuclear protein and RNA, etc., between the nucleus and the cytoplasm through nuclear pore complexes (NPCs), plays an important role in developmental processes, signal transductions and gene expression regulations. In general, ions and small proteins (namely those having a molecular weight in the range of about 40 to 60 kDa) can pass through the NPCs and enter the cell nucleus via passive diffusion. However, for large proteins to pass through the NPCs and enter the cell nucleus, nuclear localization signal (NLS)-mediated active transport is necessarily required.

Typically, most of the NLSs are short peptides that contain one or two clusters of basic amino acid residues. NLSs can be recognized by a member in the family of importins (which act as a carrier and include, e.g., importin α and importin β) to thereby trigger the binding of the importin with a substrate protein, leading said substrate protein to be imported into the cell nucleus by passing through the NPCs.

Currently, NLSs may be divided into three groups as follows:
(i) monopartite NLSs, which are primarily constituted of one cluster of basic amino acid residues, such as lysine and arginine, and a representative of which is the SV40 large T-antigen NLS (PKKKRKV; SEQ ID NO: 1);
(ii) bipartite NLSs, which are primarily constituted of two clusters of basic amino acid residues separated by a spacer of about 10˜12 amino acid residues, and a representative of which is the nucleoplasmin NLS (KRPAATKKAGQAKKKK; SEQ ID NO: 2); and
(iii) noncanonical NLSs, which are primarily constituted of polar or nonpolar amino acid residues, and representatives of which include, e.g., the M9 domain of the hnRNP A1 protein, the NLS in the influenza virus nucleoprotein, the NLS in the yeast transcription repressor Matα2, and the NLS in the yeast Ga14 protein.

It is previously reported that positively-charged NLS peptides can be coupled to negatively-charged DNA molecules via electrostatic interactions to thereby enhance the nuclear transport of said DNA molecules. Alternatively, the NLS peptides can be covalently coupled to either a condensing agent (such as a cationic polymer) of a gene delivery system or the phosphate backbone of a DNA molecule (Marieke A. E. M. van der Aa et al. (2006), Pharmaceutical Research, 23:447-459). In addition, the NLS peptides can be linked to an antitumor drug (such as a photosensitizer or a radionuclide) to deliver said antitumor drug into the cell nucleus for therapy (T. V. Akhlynina et al. (1997), J. Biol. Chem., 272:20328-20331; A. S. Sobolev (2009), Biochemistry (Moscow), 74:1567-1574).

In view of their advantageous bioactivities as described above, NLS peptides have been widely used in gene transfection (such as the expression regulation of endogenous or exogenous nucleic acids as well as epigenetic regulation), gene therapy and drug delivery.

However, whether or not a NLS peptide would have a potential in any of the applications as described above will depend on its nuclear transport efficiency. Moreover, in order to efficiently deliver a desired substance (such as a target gene, protein, drug and the like) into the cell nucleus of a target cell and exert activity/function there, in addition to optimizing an NLS peptide in terms of nuclear transport efficiency, it is necessary to consider the bioproperties of a nuclear transport system used to deliver said substance, including cellular uptake (such as endocytosis), intracellular trafficking, etc. Accordingly, researchers in the relevant art are endeavoring to explore new NLS peptides that exhibit nuclear localization ability in mammalian cells.

Chicken anemia virus (CAV), also called “chicken infectious anemia virus (CIAV)”, is a small non-enveloped, single-stranded, circular DNA virus that causes a severe immunosuppressive syndrome and anemia in infected chickens. Up to the present, a large number of CAV isolates, including strains from Australia, Bangladesh, Brazil, China, Germany, Malaysia, Nigeria, Slovenia, Taiwan and USA, have been reported and have had full or partial sequences published (Schat K A (2009), Curr Top Microbiol Immunol., 331:151-183; and Y. S. Lu et al. (1993), Exp. Rep. TPRIAH, 29:81-89).

The DNA genome of CAV is about 2.3 kb in size and there are three open reading frames (ORFs) present on the negative sense genome. At least three viral proteins are produced from a single polycistronic 2.1 kb mRNA that is produced as a single molecule and contains a promoter, TATA-box, and poly (A) signal. The three translated proteins are called VP1, VP2 and VP3, respectively, in which VP1 is a 51 kDa protein that is the structure protein involved in assembly of the viral caspid; VP2 is a 24 kDa protein that contains a dual-specificity phosphatase (DSP) activity and is required for virus infection, assembly and replication; and VP3, also called apoptin, is a 13 kDa protein that induces apoptosis in infected chicken cells.

It has been reported that the CAV VP3 protein contains two NLSs, one (i.e., NLS1) being located at positions spanning amino acid residues 82 to 88, and the other (i.e., NLS2) being located at positions spanning amino acid residues 111 to 121, in which these two NLSs together act as a bipartite NLS and constitute a tumor cell-specific nuclear targeting signal that enables the VP3 protein to specifically induce apoptosis in tumor and transformed cells but not in normal or untransformed cells (Astrid A. A. M. Danen-van Oorschot et al. (2003), J. Biol. Chem., 278: 27729-27736; Ivan K. H. Poon et al. (2005), Cancer Res., 65:7059-7064).

C. Lacorte et al. assessed the expression of three green fluorescent protein (GFP)-fused CAV proteins, namely GFP:VP1, GFP:VP2 and GFP:VP3, in plant cells. VP1, VP2 and VP3 fused to GFP all showed nuclear localization, indicating that nuclear localization signals of these three CAV proteins were functional in plants. However, this nuclear localization is not always observed, since VP3 does not localize in the nucleus of normal human cells (C. Lacorte et al. (2007), Virus Research, 129:80-86).

However, in view of the short length and sequence divergence of NLS peptides, it is difficult to predict the location of functional NLS peptide(s) in a specific protein simply by analyzing the amino acid sequence of said protein.

Insofar as the applicants know, the specific mechanism for the nuclear localization of the CAV VP2 protein has yet to be understood. In this invention, the applicants endeavored to explore NLS peptide(s) from the CAV VP2 protein that is/are functional in mammalian cells. The applicants therefore used an in silico method to analyze the VP2 proteins of various isolated strains of CAV and to predict the possible NLS peptide(s) contained therein, followed by conducting deletion analysis and point mutation analysis. The obtained results reveal that the CAV VP2 protein contains a functional NLS peptide, which is located at a region spanning amino acid residues 133-138 of the full-length amino acid sequence of the CAV VP2 protein, and which has been proved to exhibit nuclear localization ability for the nuclear delivery of functional molecules in mammalian cells.

### SUMMARY OF THE INVENTION

Therefore, in a first aspect, this invention provides an isolated peptide having nuclear localization activity, wherein the isolated peptide has an amino acid sequence that:
(i) corresponds to that of a wild-type CAV VP2 protein having 216 amino acids in length, except that amino acid residues at positions 133 and/or 134 of the wild-type CAV VP2 protein are replaced to alanine, or amino acid residues at positions 136-138 of the wild-type VP2 protein are replaced to alanine, or amino acid residues at positions 150-152 of the wild-type VP2 protein are replaced to alanine, or amino acid residues at positions 136-138 and 150-152 of the wild-type VP2 protein are replaced to alanine; or
(ii) corresponds to that of a C-terminally truncated product of the wild-type CAV VP2 protein, in which amino acid residues at positions 133-138 of the wild-type CAV VP2 protein are unchanged after C-terminal truncation; or
(iii) corresponds to that of a N-terminally truncated product of the wild-type CAV VP2 protein, in which amino acid residues at positions 133-138 of the wild-type CAV VP2 protein are unchanged after N-terminal truncation; or
(iv) corresponds to that of a N-terminally and C-terminally truncated product of the wild-type CAV VP2 protein, in which amino acid residues at positions 133-138 of the wild-type CAV VP2 protein are unchanged after N-terminal and C-terminal truncations; or
(v) is represented by formula (I):

Lys-Arg-Ala-X₁—X₂—X₃—Z (I)
wherein:
X₁, X₂ and X₃ independently represent an amino acid selected from Ala, Lys and Arg; and
Z is absent or represents Leu, Leu-Asp or Leu-Asp-Tyr.

According to a second aspect, this invention provides a nuclear transport system comprising a target substance to be delivered into the nucleus of a mammalian cell, wherein the target substance is associated with an isolated peptide as described above.

According to a third aspect, this invention provides a nucleic acid construct encoding a fusion protein comprising an isolated peptide as described above and a target protein to be delivered into the nucleus of a mammalian cell, wherein the nucleic acid construct comprises a first nucleic acid fragment encoding the isolated peptide, and a second nucleic acid fragment fused with the first nucleic acid fragment and encoding the target protein.

According to a fourth aspect, this invention provides an expression cassette capable of expressing a fusion protein comprising an isolated peptide as described above and a target protein to be delivered into the nucleus of a mammalian cell, wherein the expression cassette comprises the nucleic acid construct as described above and a promoter operably linked to the nucleic acid construct.

According to a fifth aspect, this invention provides a recombinant vector carrying the expression cassette as described above.

### BRIEF DESCRIPTION OF THE DRAWINGS

The above and other objects, features and advantages of this invention will become apparent with reference to the following detailed description and the preferred embodiments taken in conjunction with the accompanying drawings, in which:

FIG. 1 shows the construct of plasmid pGEX-6P-1-VP2, in which P_{tac} represents a tac promoter; GST represents a gene encoding glutathione S-transferase; Amp^{r} represents an ampicillin-resistance gene; vp2 represents a gene that encodes a VP2 protein of the CAV Taiwan CIA-89 strain; and EcoRI and XhoI represent the recognition sites of the corresponding restriction enzymes, respectively;

FIG. 2 shows the construct of plasmid pcDNA3.1-GFP, in which P_{CMV} represents a CMV promoter; gfp represents a gene that encodes a green fluorescent protein (GFP); Amp^{r} represents an ampicillin-resistance gene; and EcoRI and XhoI represent the recognition sites of the corresponding restriction enzymes, respectively;

FIG. 3 shows the construct of a recombinant plasmid pVP2-yT&A as obtained in Example 1, infra, in which Amp^{r} represents an ampicillin-resistance gene; vp2 represents a vp2 gene of SEQ ID NO: 3 (see Example 1, infra); and EcoRI and XhoI represent the recognition sites of the corresponding restriction enzymes, respectively;

FIG. 4 shows the construct of a recombinant plasmid pcDNA3.1-VP2-GFP as obtained in Example 1, infra, in which P_{CMV} represents the CMV promoter shown in FIG. 2; vp2 represents the vp2 gene of SEQ ID NO: 3 shown in FIG. 3; gfp represents the GFP-encoding gene shown in FIG. 2; Amp^{r} represents the ampicillin-resistance gene shown in FIG. 2; and EcoRI and XhoI represent the recognition sites of the corresponding restriction enzymes, respectively;

FIG. 5 shows the expression of GFP or VP2-GFP in HeLa cells (upper part) or CHO cells (lower part) after transfection with a control plasmid pcDNA3.1-GFP or the recombinant plasmid pcDNA3.1-VP2-GFP as obtained in Example 1, infra, as observed at visible light or at a wavelength of 480 nm (for fluorescence image) or 350 nm (for DAPI image) using a Zeiss AxioVert 200 inverted microscope under 400× magnification, in which the visible light images show the cellular morphology of the cells after transfection; the location of GFP is indicated by the emitted green fluorescence in a fluorescence image, and the location of a cell nucleus is indicated by the emitted blue fluorescence in a DAPI image;

FIG. 6 shows the amino acid sequence alignment results of the VP2 proteins of six different isolated strains of CAV, as analyzed by the Biology Workbench 3.2 software (San Diego Supercomputer Center (SDSC), San Diego, Calif., USA), in which a region of underlined amino acid residues in a VP2 protein's sequence indicates the location of a putative bipartite NLS motif (referred to as “BiNLS1 motif” hereinafter) as predicted by the WoLF PSORT software (P. Horton et al. (2007), Nucleic Acids Research, 35:W585-587), and a region of boldfaced amino acid residues in a VP2 protein's sequence indicates the location of a putative monopartite NLS motif (referred to as “NLS2 motif” hereinafter) as predicted by the NLStradamus software (Alex N Nguyen Ba et al. (2009), BMC Bioinformatics, 10:202-212);

FIG. 7 schematically shows a full-length VP2-GFP fusion protein as generated in Example 1, infra, and six truncated VP2-GFP fusion proteins as generated in Example 3, infra, in which a full-length or truncated VP2 protein is indicated by a black zone; each numeral above every black zone represents a corresponding amino acid position in the full-length VP2 protein; and a GFP protein is indicated by a white zone;

FIG. 8 shows the microscopic examination results of HeLa cells and CHO cells after transfection with six different recombinant plasmids constructed in Example 3, infra, as observed at a wavelength of 480 nm (for fluorescence image) or 350 nm (for DAPI image) using a Zeiss AxioVert 200 inverted microscope under 400× magnification, in which the six recombinant plasmids, as represented by VP2-115dC, VP2-132dC, VP2-145dC, VP2-111dN, VP2-141dN and VP2-160dN, respectively carried a truncated vp2-gfp fusion gene encoding one of the six truncated VP2-GFP fusion proteins shown in FIG. 7; the location of GFP is indicated by the emitted green fluorescence in a fluorescence image, and the location of a cell nucleus is indicated by the emitted blue fluorescence in a DAPI image;

FIG. 9 shows the microscopic examination results of HeLa cells and CHO cells after transfection with six different recombinant plasmids constructed in Example 4, infra, as observed at a wavelength of 480 nm (for fluorescence image) or 350 nm (for DAPI image) using a Zeiss AxioVert 200 inverted microscope under 400× magnification, in which the six recombinant plasmids respectively carried a mutant vp2-gfp fusion gene encoding a mutant VP2-GFP fusion protein that contained a mutant VP2 protein represented by VP2-150-152A, VP2-136-138A, VP2-136-138A/150-152A, VP2-136-138A/133A, VP2-136-138A/134A or VP2-136-138A/133A/134A; the location of GFP is indicated by the emitted green fluorescence in a fluorescence image, and the location of a cell nucleus is indicated by the emitted blue fluorescence in a DAPI image;

FIG. 10 shows the microscopic examination results of HeLa cells after transfection with three different recombinant plasmids constructed in Example 4, infra, as observed at a wavelength of 480 nm (for fluorescence image) or 350 nm (for DAPI image) using a Zeiss AxioVert 200 inverted microscope under 400× magnification, in which the three recombinant plasmids respectively carried a mutant vp2-gfp fusion gene encoding a mutant VP2-GFP fusion protein that contained a mutant VP2 protein represented by VP2-133A, VP2-134A or VP2-133A/134A; the location of GFP is indicated by the emitted green fluorescence in a fluorescence image, and the location of a cell nucleus is indicated by the emitted blue fluorescence in a DAPI image;

FIG. 11 shows the microscopic examination results of HeLa cells and CHO cells after transfection with a recombinant plasmid pcDNA3.1-VP2 (112-145)-GFP (represented by “VP2 (112-145)”) or a recombinant plasmid pcDNA3.1-VP2 (133-138)-GFP (represented by “VP2 (133-138)”) as constructed in Example 5, infra, as observed at visible light or at a wavelength of 480 nm (for fluorescence image) or 350 nm (for DAPI image) using a Zeiss AxioVert 200 inverted microscope under 400× magnification, in which the visible light images show the cellular morphology of the cells after transfection; the location of GFP is indicated by the emitted green fluorescence in a fluorescence image, and the location of a cell nucleus is indicated by the emitted blue fluorescence in a DAPI image; and

FIG. 12 shows the microscopic examination results of HeLa cells after transfection with the recombinant plasmid pcDNA3.1-VP2 (112-145)-GFP (represented by “VP2 (112-145)”) constructed in Example 5, infra, and four recombinant plasmids pcDNA3.1-VP2 (112-145)-136-138A-GFP (represented by “VP2 (112-145)-136-138A”), pcDNA3.1-VP2 (112-145)-136-138A/133A-GFP (represented by “VP2 (112-145)-136-138A/133A”), pcDNA3.1-VP2 (112-145)-136-138A/134A-GFP (represented by “VP2 (112-145)-136-138A/134A”) and pcDNA3.1-VP2 (112-145)-136-138A/133A/134A-GFP (represented by “VP2 (112-145)-136-138A/133A/134A”) constructed in Example 6, infra, as observed at visible light or at a wavelength of 480 nm (for fluorescence image) using a Zeiss AxioVert 200 inverted microscope under 400× magnification, in which the location of GFP is indicated by the emitted green fluorescence in a fluorescence image, and a merge image represents a merger of the fluorescence image and a corresponding visible light image that shows the cellular morphology of the cells after transfection.

### DETAILED DESCRIPTION OF THE INVENTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprise(s)” has a corresponding meaning.

Unless defined otherwise, all technical and scientific terms used herein have the meaning commonly understood by a person skilled in the art to which this invention belongs. One skilled in the art will recognize many methods and materials similar or equivalent to those described herein, which could be used in the practice of the present invention. Indeed, the present invention is in no way limited to the methods and materials described. For clarity, the following definitions are used herein.

Unless otherwise indicated, nucleic acids are written left to right in 5′ to 3′ orientation and amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The above-defined terms are more fully defined by reference to the instant Specification as a whole.

“Recombinant DNA technology” refers to techniques for uniting two heterologous DNA molecules, usually as a result of in vitro ligation of DNAs from different organisms. Recombinant DNA molecules are commonly produced by experiments in genetic engineering. Synonymous terms include “gene splicing,” “molecular cloning” and “genetic engineering.” The product of these manipulations results in a “recombinant” or “recombinant molecule.”

Techniques for manipulating nucleic acids, such as those for generating mutation in sequences, subcloning, labeling, probing, sequencing, hybridization and so forth, are described in detail in scientific publications and patent documents. See, for example, Sambrook J, Russell D W (2001) Molecular Cloning: a Laboratory Manual, 3rd ed. Cold Spring Harbor Laboratory Press, New York; Current Protocols in Molecular Biology, Ausubel ed., John Wiley & Sons, Inc., New York (1997); and Laboratory Techniques in Biochemistry and Molecular Biology: Hybridization With Nucleic Acid Probes, Part I, Theory and Nucleic Acid Preparation, Tijssen ed., Elsevier, N.Y. (1993).

As used herein, the term “derived from” refers to a component that is isolated from or made using a specified molecule or organism, or information from the specified molecule or organism. For example, a polypeptide that is derived from a second polypeptide can include an amino acid sequence that is identical or substantially similar to the amino acid sequence of the second polypeptide. In the case of polypeptides, the derived species can be obtained by, for example, naturally occurring mutagenesis, artificial directed mutagenesis or artificial random mutagenesis. The mutagenesis used to derive polypeptides can be intentionally directed or intentionally random, or a mixture of each. The mutagenesis of a polypeptide to create a different polypeptide derived from the first can be a random event (e.g., caused by polymerase infidelity) and the identification of the derived polypeptide can be made by appropriate screening methods, e.g., as discussed herein. Mutagenesis of a polypeptide typically entails manipulation of the polynucleotide that encodes the polypeptide. As used herein, the term “derived from” encompasses the terms “originated from,” “obtained or obtainable from,” and “isolated from.”

As used herein, the terms “isolated and/or purified” refer to in vitro preparation, isolation and/or purification of a nucleic acid molecule, a polypeptide, peptide or protein, so that it is not associated with in vivo substances. Thus, the term “isolated” when used in relation to a nucleic acid, as in “isolated oligonucleotide” or “isolated polynucleotide” refers to a nucleic acid sequence that is identified and separated from at least one contaminant with which it is ordinarily associated in its source. An isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; and RNA sequences (e.g., a specific mRNA sequence encoding a specific protein) are found in the cell as a mixture with numerous other mRNAs that encode a multitude of proteins. Hence, with respect to an “isolated nucleic acid molecule,” which includes a polynucleotide of genomic, cDNA, or synthetic origin or some combination thereof, the “isolated nucleic acid molecule” (1) is not associated with all or a portion of a polynucleotide in which the “isolated nucleic acid molecule” is found in nature, (2) is operably linked to a polynucleotide which it is not linked to in nature, or (3) does not occur in nature as part of a larger sequence. The isolated nucleic acid molecule may be present in single-stranded or double-stranded form.

As used herein, the term “target substance” refers to a substance which is intended to be introduced into the nucleus of a cell. Substances targeted by this invention are substances which are not introduced under normal conditions. Therefore, substances which can be introduced into cells by diffusion or hydrophobic interaction under normal conditions are not targeted in an important aspect of this invention. Examples of substances which are not introduced into cells under normal conditions include, but are not limited to, proteins (polypeptides), RNA, DNA, polysaccharides, and composite molecules thereof (e.g., glycoproteins, PNA, etc.), viral vectors, and other compounds.

As used herein, the term “associated with” describes the interaction between or among two or more groups, moieties, compounds, monomers, etc. When two or more entities are “associated with” one another as described herein, they are linked by a direct or indirect covalent or non-covalent interaction. Preferably, the association is covalent. The covalent association may be, for example, but without limitation, through an amide, ester, carbon-carbon, disulfide, carbamate, ether, thioether, urea, amine, or carbonate linkage. The covalent association may also include a linker moiety, e.g., a spacer sequence that links two polypeptide molecules. Desirable non-covalent interactions include hydrogen bonding, van der Waals interactions, dipole-dipole interactions, pi stacking interactions, hydrophobic interactions, magnetic interactions, electrostatic interactions, etc. Also, two or more entities or agents may be “associated with” one another by being present together in the same composition. As used herein, the term “associated with” may be synonymous with the terms “bound to,” “coupled to,” “linked to,” “attached with,” “conjugated with,” “fused with,” etc.

As used herein, the term “conjugate” or “conjugation” refers to the attachment of two or more compounds, in particular proteins, joined together to form one entity. These compounds may be attached together by linker moieties, chemical modification, peptide linkers, chemical linkers, covalent or non-covalent bonds, or protein fusion or by any means known to one skilled in the art. The joining may be permanent or reversible. In some embodiments, several linkers may be included in order to take advantage of desired properties of each linker and each protein in the conjugate. Flexible linkers and linkers that increase the solubility of the conjugates are contemplated for use alone or with other linkers are incorporated herein. Peptide linkers may be linked by expressing DNA encoding the linker to one or more proteins in the conjugate. Linkers may be acid cleavable, photocleavable and heat-sensitive linkers.

The term “sequence divergence” as used herein refers to the percent difference in the nucleotide sequence in a comparison between related nucleic acid sequences, or in the amino acid sequence in a comparison between related proteins.

The term “% identity” as used herein refers to the level of identity between two amino acid or nucleic acid sequences, as determined by a defined algorithm, and accordingly a homologue of a given sequence has at least about 70% or 80%, preferably about 90, 95 or 98% sequence identity over a length of the given sequence. It will be understood that the term “70% homology” means the same thing as 70% sequence identity.

As used herein, the terms “protein,” “polypeptide” and “peptide” can be used interchangeably, and refer to an organic polymer composed of two or more constituent amino acids that are connected via peptide bonds or other bonds such as ester bonds, ether bonds, etc. As used herein, the term “protein” typically refers to large polypeptides, and the term “peptide” typically refers to short polypeptides. As used herein, the term “amino acid” refers to either the natural and/or non-natural or synthetic amino acids, including glycine and both the D and L optical isomers, amino acid analogs and peptidomimetics.

As used herein, the term “corresponding to” or “corresponds to” is often used to designate the position/identity of an amino acid residue in a polypeptide. Those of ordinary skill will appreciate that, for purposes of simplicity, a canonical numbering system is typically used to designate positions in a polypeptide with reference to a particular established reference polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art may readily appreciate how to identify corresponding amino acids. The definition of the term “corresponding to” also applies to the nucleotide residues in a nucleic acid molecule.

The term “recombinant polypeptide” as used herein is defined as a polypeptide produced by recombinant DNA methodologies, in which the polypeptide is produced upon expression of a recombinant polynucleotide encoding the same. Alternatively, polypeptides may be synthesized chemically, for example, using an automated polypeptide synthesizer.

As used herein, the term “wild-type” refers to a gene or gene product which has the characteristics of that gene or gene product when isolated from a naturally-occurring source. As used herein, the term “wild-type” is used interchangeably with the term “naturally-occurring.”

A “wild-type” protein means that the protein will be active at a level of activity found in nature and typically will be the amino acid sequence found in nature. In an aspect, the term “wild type” or “parental sequence” can indicate a starting or reference sequence prior to a manipulation of this invention.

As used herein, the term “mutation” refers to a change introduced into a parental sequence, including, but not limited to, substitutions, insertions, and deletions (including truncations). The consequences of a mutation include, but are not limited to, the creation of a new character, property, function, phenotype or trait not found in the protein encoded by the parental sequence.

As used herein, the term “mutant” refers to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product. The mutant may be one that exists in nature, such as an allelic mutant, or one not yet identified in nature. The mutant may be conservatively altered, wherein substituted amino acid(s) retain structural or chemical characteristics similar to those of the original amino acid(s). Rarely, mutants may be substituted non-conservatively.

As used herein, the term “substitution” refers to the replacement of one or more amino acids or nucleotides by different amino acids or nucleotides, respectively, as compared to the naturally occurring molecule.

The term “C-terminally truncated product” with reference to a protein, polypeptide or fragment thereof generally denotes such product that has a C-terminal deletion of one or more amino acid residues as compared to said protein, polypeptide or fragment thereof.

The term “N-terminally truncated product” with reference to a protein, polypeptide or fragment thereof generally denotes such product that has an N-terminal deletion of one or more amino acid residues as compared to said protein, polypeptide or fragment thereof.

The terms “nucleic acid” and “nucleic acid sequence” as used herein refer to a deoxyribonucleotide or ribonucleotide sequence in single-stranded or double-stranded form, that comprises naturally occurring and known nucleotides or artificial chemical mimics. The term “nucleic acid” as used herein is interchangeable with the terms “gene,” “cDNA,” “mRNA,” “oligonucleotide” and “polynucleotide” in use.

As used herein, the term “polynucleotide” refers to a sequence of nucleotides connected by phosphodiester linkages. A polynucleotide of this invention can be a deoxyribonucleic acid (DNA) molecule or ribonucleic acid (RNA) molecule in either single- or double-stranded form. Nucleotide bases are indicated herein by a single letter code: adenine (A), guanine (G), thymine (T), cytosine (C), inosine (I) and uracil (U). A polynucleotide of this invention can be prepared using standard techniques well known to one of ordinary skill in the art. This term is not to be construed as limiting with respect to the length of a polymer, and encompasses known analogues of natural nucleotides, as well as nucleotides that are modified in the sugar and/or phosphate moieties. This term also encompasses nucleic acids containing modified backbone residues or linkages, which are synthetic, naturally occurring, and non-naturally occurring, which have similar binding properties as the reference nucleic acid, and which are metabolized in a manner similar to the reference nucleotides.

As used herein, the term “DNA fragment” may be used interchangeably with the term “nucleic acid fragment” and refers to a DNA polymer, in the form of a separate segment or as a component of a larger DNA construct, which has been derived either from isolated DNA or synthesized chemically or enzymatically such as by methods disclosed elsewhere.

As used herein, the term “gene” refers to a DNA sequence, including but not limited to a DNA sequence that can be transcribed into mRNA which can be translated into polypeptide chains, transcribed into rRNA or tRNA, or serve as recognition sites for enzymes and other proteins involved in DNA replication, transcription and regulation. This definition includes various sequence polymorphisms, mutations, and/or sequence variants wherein such alterations do not affect the function of the gene product. The term “gene” is intended to include not only regions encoding gene products but also regulatory regions including, e.g., promoters, termination regions, translational regulatory sequences (such as ribosome binding sites and internal ribosome entry sites), enhancers, silencers, insulators, boundary elements, replication origins, matrix attachment sites, and locus control regions. The term “gene” further includes all introns and other DNA sequences spliced from the mRNA transcript, along with variants resulting from alternative splice sites. The term “gene” includes, but is not limited to, structural genes, immunity genes and secretory (transport) genes.

As used herein, the term “fusion gene” refers to a DNA fragment in which two or more genes are fused in a single reading frame to encode two or more proteins that are fused together via one or more peptide bonds. As used herein, the term “fusion protein” refers to a protein or polypeptide encoded by a fusion gene and it may be used interchangeably with the term “fusion gene product.”

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The term “codon” as used herein, is a basic genetic coding unit, consisting of a sequence of three nucleotides that specify a particular amino acid to be incorporated into a polypeptide chain, or a start or stop signal. The term “coding region” when used in reference to structural gene refers to the nucleotide sequences that encode the amino acids found in the nascent polypeptide as a result of translation of a mRNA molecule.

A DNA “coding sequence” is a double-stranded DNA sequence which is transcribed into RNA, and the RNA is translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A DNA coding sequence can include, but is not limited to, prokaryotic sequences, sequences from the genomes of viruses that infect prokaryotes or eukaryotes, cDNA from eukaryotic mRNA, genomic DNA sequences from eukaryotic (e.g., mammalian) DNA, and even synthetic DNA sequences. A polyadenylation signal and transcription termination sequence are usually located downstream of the coding sequence. A “cDNA” is defined as copy-DNA or complementary-DNA, and is a product of a reverse transcription reaction from a mRNA transcript.

As used herein, the term “isolated DNA” denotes that the DNA has been removed from its natural genetic environment and is thus free of other extraneous or undesired coding sequences, and is in a form suitable for use within genetically engineered protein production systems. The “isolated DNA” may be synthesized by chemical processes, recombinant DNA technology or by the conventional techniques commonly employed in the field of biotechnology, such as DNA shuffling experiments or site-directed mutagenesis experiments. The term “an isolated DNA” is alternatively termed “a cloned DNA.”

Unless otherwise indicated, a nucleic acid sequence, in addition to the specific sequences described herein, also covers its complementary sequence, and the conservative analogs, related naturally occurring structural variants and/or synthetic non-naturally occurring analogs thereof, for example, homologous sequences having degenerative codon substitution, and conservative deletion, insertion, substitution, or addition. Specifically, degenerative codon substitution may be produced by, for instance, a nucleotide residue substitution at the third position of one or more selected codons in a nucleic acid sequence with other nucleotide residue(s).

The term “nucleic acid construct” as used herein refers to a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a coding sequence of this invention.

As used herein, the term “expression cassette” refers to a construct of genetic material that contains a coding sequence and enough regulatory information to direct proper transcription and translation of the coding sequence in a recipient cell. The expression cassette may be inserted into a vector for targeting to a desired host cell and/or into a subject.

The term “expression vector” as used herein refers to any recombinant expression system capable of expressing a selected nucleic acid sequence, in any host cell in vitro or in vivo, constitutively or inducibly. The expression vector may be an expression system in linear or circular form, and covers expression systems that remain episomal or that integrate into the host cell genome. The expression system may or may not have the ability to self-replicate, and it may drive only transient expression in a host cell. Typically, an expression vector contains an origin of replication which is functional in host cells, and selectable markers for detecting host cells comprising the expression vector. Expression vectors of this invention contain a promoter sequence and include genetic elements as described herein arranged such that an inserted coding sequence can be transcribed and translated in host cells. In certain embodiments described herein, an expression vector is a closed circular DNA molecule. The term “expression vector” is interchangeable with the terms “recombinant vector,” “plasmid” and “recombinant plasmid” in use.

As used herein, the term “promoter” can be used interchangeably with the term “promoter sequence” and refers to a DNA regulatory region capable of binding RNA polymerase in a cell and initiating transcription of a downstream (3′ direction) coding sequence. The promoter is bound at its 3′ terminus by the translation start codon of a coding sequence and extends upstream (5′ direction) to include a minimum number of bases or elements necessary to initiate transcription. Promoters which cause a gene to be expressed in most cell types at most times are commonly referred to as “constitutive promoters”. Promoters which cause conditional expression of a structural nucleotide sequence under the influence of changing environmental conditions or developmental conditions are commonly referred to as “inducible promoter.” Promoter sequences suitable for use in this invention may be derived from viruses, bacteriophages, prokaryotes or eukaryotes.

The term “operably linked” as used herein means that a first sequence is disposed sufficiently close to a second sequence such that the first sequence can influence the second sequence or regions under the control of the second sequence. For instance, a promoter sequence may be operably linked to a gene sequence, and is normally located at the 5′-terminus of the gene sequence such that the expression of the gene sequence is under the control of the promoter sequence. In addition, a regulatory sequence may be operably linked to a promoter sequence so as to enhance the ability of the promoter sequence in promoting transcription. In such case, the regulatory sequence is generally located at the 5′-terminus of the promoter sequence.

As used herein, the term “upstream” and “downstream” refer to the position of an element of nucleotide sequence. “Upstream” signifies an element that is more 5′ than the reference element. “Downstream” signifies an element that is more 3′ than the reference element.

According to this invention, the term “transformation” can be used interchangeably with the term “transfection” when such term is used to refer to the introduction of an exogenous nucleic acid molecule into a selected host cell. According to techniques known in the art, a nucleic acid molecule (e.g., a recombinant DNA construct or a recombinant vector) can be introduced into a selected host cell by various techniques, such as calcium phosphate- or calcium chloride-mediated transfection, electroporation, microinjection, particle bombardment, liposome-mediated transfection, transfection using bacterial bacteriaphages, or other methods. Host organisms containing the transformed nucleic acid molecule are referred to as “transformed” or “transgenic” or “recombinant” organisms.

The terms “cell,” “host cell,” “transformed host cell” and “recombinant host cell” as used herein can be interchangeably used, and not only refer to specific individual cells but also include sub-cultured offsprings or potential offsprings thereof. Sub-cultured offsprings formed in subsequent generations may include specific genetic modifications due to mutation or environmental influences and, therefore, may factually not be fully identical to the parent cells from which the sub-cultured offsprings were derived. However, sub-cultured cells still fall within the coverage of the terms used herein.

As used herein, the term “mammalian cell” includes cells that are derived from a normal or tumorous tissue of a mammal. According to this invention, the mammal may be selected from the group consisting of humans, bovine, sheep, goats, horses, dogs, cats, rabbits, rats, and mice.

“Nuclear localization signal (NLS)” is a specific peptide motif or segment present in a variety of proteins characterized by its capacity to direct the protein to the nucleus of a cell. In view of their ability to transport a target substance such as a protein or polynucleotide into the nucleus of a cell, NLSs are contemplated to have a wide range of utilities, including, e.g., gene transfection, gene therapy, drug delivery, etc. Interestingly, most NLSs do not consist of a consensus sequence, although the NLS of SV40 large T antigen provides the prototypic monopartite NLS. Accordingly, researchers in the relevant art are endeavoring to explore novel and useful NLSs from various proteins of different organisms.

While the native full-length VP2 protein of chicken anemia virus (referred to as “CAV VP2 protein” hereinafter) has been reported to exhibit nuclear localization function, it has yet to be known any NLS peptide in the CAV VP2 protein that is functional in mammalian cells. In this invention, the applicants verified the nuclear localization ability of the VP2 protein of the CAV Taiwan CIA-89 strain (Meng-Shiou Lee et al. (2009), Process Biochemistry, 44:390-395) in two mammalian cell lines, i.e., HeLa cells and CHO cells, and then analyzed the amino acid sequence of the VP2 protein of the CAV Taiwan CIA-89 strain, as compared to those of the VP2 proteins of a number of isolated strains of CAV as deposited in the UniProtKB database, including:

Australia/CAU269-7/2000 (UniProtKB Accession Number: Q9IZU7),

Germany Cuxhaven-1 (UniProtKB Accession Number: P69484),

Japan 82-2 (UniProtKB Accession Number: P54093),

USA 26p4 (UniProtKB Accession Number: P54092), and

USA CIA-1 (UniProtKB Accession Number: P69485).

Based on the obtained sequence alignment results, which reveal that the amino acid sequence of the CAV VP2 protein is highly conserved in different isolated strains, the applicants used the WoLF PSORT software (P. Horton et al. (2007), supra) and the NLStradamus software (Alex N Nguyen Ba et al. (2009), supra) to explore NLS peptide(s) in the full-length amino acid sequence of the VP2 protein of the CAV Taiwan CIA-89 strain, in which a bipartite NLS motif (BiNLS1 motif; SEQ ID NO: 4) was predicted to be located at a position spanning amino acid residues 136-152 of the CAV VP2 protein, and a monopartite NLS motif (NLS2 motif; SEQ ID NO: 5) was predicted to be located at a position spanning amino acid residues 133-138 of the CAV VP2 protein.

To verify these two predicted NLS motifs, the applicants constructed a series of recombinant plasmids, each carrying a fusion gene encoding a C-terminal or N-terminal truncated VP2-GFP fusion protein. The obtained expression results reveal that when the CAV VP2 protein was C-terminally truncated to a length containing amino acids residues 1-132, or N-terminally truncated to a length containing amino acid residues 142-216, its nuclear localization ability would be abrogated, suggesting that a NLS peptide of SEQ ID NO: 6, which fully covered the predicted NLS2 motif, might be located at a region spanning amino acid residues 133-141 of the CAV VP2 protein.

In view of the possible locations of the putative BiNLS1 and NLS2 motifs in the CAV VP2 protein, the applicants further conducted various site-directed mutations at amino acid positions 133-134, 136-138 and 150-152 of the CAV VP2 protein where basic amino acid residues were located, so as to evaluate the criticality of these basic amino acid residues to the CAV VP2 protein in terms of nuclear localization ability. The obtained expression results reveal that alanine substitutions at amino acid positions 136-138 of the CAV VP2 protein, or alanine substitutions at amino acid positions 150-152 of the CAV VP2 protein, or alanine substitutions at amino acid positions 136-138 and 150-152 of the CAV VP2 protein, or alanine substitutions at amino acid positions 133 and/or 134 of the CAV VP2 protein, did not abrogate the CAV VP2 protein's nuclear localization ability, indicating that the predicted BiNLS1 motif was not the functional NLS peptide contained in the CAV VP2 protein and that a functional NLS peptide should be located at a region spanning amino acid residues 133 to 138 of the CAV VP2 protein, which region was matched with the location of the predicted NLS2 motif of SEQ ID NO: 5. In addition, the amino acid residues at positions 133-134 and/or 136-138 might be critical to the nuclear localization ability of the CAV VP2 protein.

To verify the role of the predicted NLS2 motif of SEQ ID NO: 5 in the CAV VP2 protein, the applicants further constructed two short-length peptides derived from the CAV VP2 protein, namely VP2 (133-138) and VP2 (112-145), in which the former has an amino acid sequence as shown in SEQ ID NO: 5 (i.e., the predicted NLS2 motif in full length) and corresponds to amino acid positions 133-138 of the CAV VP2 protein, and the latter has an amino acid sequence as shown in SEQ ID NO: 7 and corresponds to amino acid positions 112-145 of the CAV VP2 protein. These two short-length peptides were subjected to a nuclear transport assay using the GFP protein as a reporter, and the obtained results reveal that these two short-length peptides exhibit nuclear localization ability as that of the full-length CAV VP2 protein, indicating that an intact and functional NLS motif is present in a region spanning amino acid residues 133-138 of the CAV VP2 protein.

The applicants further constructed four mutants of the VP2 (112-145) peptide, each mutant having alanine substitutions at positions corresonding to amino acid residues 136-138, or amino acid residues 133 and 136-138, or amino acid residues 134 and 136-138, or amino acid residues 133-134 and 136-138, of the CAV VP2 protein. The obtained results reveal that the amino acid residues that correspond to amino acid positions 133-134 and 136-138 of the CAV VP2 protein might play an important role in the nuclear localization ability of the VP2 (112-145) peptide. This finding is consistent with that observed for the full-length CAV VP2 protein.

Based on the obtained experimental results, it is contemplated that a NLS peptide derived from the CAV VP2 protein can be used in the nuclear transport of a variety of biologically active substances, including nucleic acids, proteins, peptides, pharmaceutically active agents, chemical substances, etc.

Therefore, this invention provides an isolated peptide having nuclear localization activity, wherein the isolated peptide has an amino acid sequence that:
(i) corresponds to that of a wild-type CAV VP2 protein having 216 amino acids in length, except that amino acid residues at positions 133 and/or 134 of the wild-type CAV VP2 protein are replaced to alanine, or amino acid residues at positions 136-138 of the wild-type VP2 protein are replaced to alanine, or amino acid residues at positions 150-152 of the wild-type VP2 protein are replaced to alanine, or amino acid residues at positions 136-138 and 150-152 of the wild-type VP2 protein are replaced to alanine; or
(ii) corresponds to that of a C-terminally truncated product of the wild-type CAV VP2 protein, in which amino acid residues at positions 133-138 of the wild-type CAV VP2 protein are unchanged after C-terminal truncation; or
(iii) corresponds to that of a N-terminally truncated product of the wild-type CAV VP2 protein, in which amino acid residues at positions 133-138 of the wild-type CAV VP2 protein are unchanged after N-terminal truncation; or

(iv) corresponds to that of a N-terminally and C-terminally truncated product of the wild-type CAV VP2 protein, in which amino acid residues at positions 133-138 of the wild-type CAV VP2 protein are unchanged after N-terminal and C-terminal truncations; or
(v) is represented by formula (I):

Lys-Arg-Ala-X₁—X₂—X₃—Z (I)
wherein:
X₁, X₂ and X₃ independently represent an amino acid selected from Ala, Lys and Arg; and
Z is absent or represents Leu, Leu-Asp or Leu-Asp-Tyr.

According to this invention, the wild-type CAV VP2 protein may be derived from any of the following isolated strains of CAV: CAV Taiwan CIA-89 strain, CAV Australia/CAU269-7/2000 strain (UniProtKB Accession Number: Q91ZU7), CAV Germany Cuxhaven-1 strain (UniProtKB Accession Number: P69484), CAV Japan 82-2 strain (UniProtKB Accession Number: P54093), CAV USA 26p4 strain (UniProtKB Accession Number: P54092), and CAV USA CIA-1 strain (UniProtKB Accession Number: P69485). In a preferred embodiment of this invention, the wild-type CAV VP2 protein has an amino acid sequence selected from SEQ ID NO: 8, SEQ ID NO: 9, SEQ ID NO: 10, SEQ ID NO: 11, SEQ ID NO: 12 and SEQ ID NO: 13.

In a preferred embodiment of this invention, the isolated peptide has an amino acid sequence selected from SEQ ID NO: 14, SEQ ID NO: 15, SEQ ID NO: 16, SEQ ID NO: 17, SEQ ID NO: 18 and SEQ ID NO: 19.

In another preferred embodiment of this invention, the isolated peptide has an amino acid sequence corresponding to that of a C-terminally truncated product of the wild-type CAV VP2 protein. In a more preferred embodiment of this invention, the C-terminally truncated product of the wild-type CAV VP2 protein has an amino acid sequence of SEQ ID NO: 20.

In a further preferred embodiment of this invention, the isolated peptide has an amino acid sequence corresponding to that of a N-terminally truncated product of the wild-type CAV VP2 protein. In a more preferred embodiment of this invention, the N-terminally truncated product of the wild-type CAV VP2 protein has an amino acid sequence of SEQ ID NO: 21.

In a further preferred embodiment of this invention, the isolated peptide has an amino acid sequence corresponding to that of a N-terminally and C-terminally truncated product of the wild-type CAV VP2 protein. In a more preferred embodiment of this invention, the N-terminally and C-terminally truncated product of the wild-type CAV VP2 protein has an amino acid sequence of SEQ ID NO: 5, SEQ ID NO: 6 or SEQ ID NO: 7.

The isolated peptide of this invention can be chemically, enzymatically or recombinantly synthesized, or may be derived from a natural source. In a preferred embodiment of this invention, the isolated peptide is synthesized by recombinant DNA methods.

According to this invention, the isolated peptide may be synthesized as a fusion protein, in which the isolated peptide is fused with a target protein that is intended to be transported into the nucleus of a cell, in particular a mammalian cell. In a preferred embodiment of this invention, the fusion protein is synthesized by recombinant DNA methods.

This invention further provides a nuclear transport system comprising a target substance to be delivered into the nucleus of a mammalian cell, wherein the target substance is associated with an isolated peptide of this invention as described above.

According to this invention, the nuclear transport system further comprises a binding reagent that enables the nuclear transport system to enter into a mammalian cell before the target substance is transported into the nucleus of the mammalian cell. The binding reagent is one capable of binding to a specific cell surface-expressing antigen or receptor on the outer cell membrane or plasma membrane of a mammalian cell, so that the nuclear transport system can enter into the cytoplasm of the cell by endocytosis, after which the nuclear transport system is transported into the nucleus of the cell through an importin-NLS pathway. In a preferred embodiment of this invention, the binding reagent is an antibody or functional fragment thereof that binds to a specific cell surface-expressing antigen or receptor on the outer cell membrane or plasma membrane of a mammalian cell.

According to this invention, the term “target substance” is synonymous with the term “effector” and refers to any molecule or compound of interest that exhibits a desired biological activity or effect (e.g., pharmaceutical, diagnostic or tracing properties) when delivered into a cell.

According to this invention, the target substance may be selected from the group consisting of proteins, peptides, nucleic acid molecules, pharmaceutically active agents, chemical substances, lipids, carbohydrates, and combinations thereof.

Nucleic acid molecules suitable for use in this invention include, but are not limited to, DNA molecules, RNA molecules, peptide nucleic acids (PNAs), small interfering RNAs (siRNAs), antisense molecules, ribozymes, aptamers and decoy molecules.

Proteins suitable for use in this invention include, but are not limited to, enzymes, hormones, cytokines, apolipoproteins, growth factors, antigens, antibodies and antibody fragments.

Peptides suitable for use in this invention as the target substance include, but are not limited to, antigenic peptides, antimicrobial peptides and anti-inflammatory peptides.

The term “pharmaceutically active agent” as used herein refers to a chemical compound that induces a detectable pharmacological and/or physiological effect when administered to a subject. Pharmaceutically active agent suitable for use in this invention include, but are not limited to, toxins, antibiotics, antipathogenic agents, immunomodulators, vitamins, antineoplastic agents, therapeutic agents.

Chemical substances suitable for use in this invention include, but are not limited to, organic molecules, inorganic molecules, radioisotopes, fluorescent particles, magnetic particles and metal nanoparticles.

Lipids suitable for use in this invention include, but are not limited to, fatty acids, glycerolipids, phospholipids, sterol lipid and saccharolipids. Carbohydrates suitable for use in this invention include, but are not limited to, monosaccharides, disaccharides, oligosaccharides and polysaccharides.

In a preferred embodiment of this invention, the isolated peptide of this invention and the target substance together form a conjugate. In a more preferred embodiment of this invention, the target substance is conjugated with the isolated peptide of this invention via a linker moiety, which may be a chemical linker, a spacer sequence composed of amino acids, etc. Preferably, the linker moiety provides a strong linkage between the isolated peptide of this invention and the target substance to prevent dissociation of the two during the transport of the target substance into the nucleus of a mammalian cell. In a preferred embodiment of this invention, the target substance is a protein or polypeptide and the linker moiety is a spacer sequence composed of amino acids.

According to this invention, the isolated peptide and the target substance can be separately synthesized by conventional chemical processes, e.g., using a commercially available synthesis kit or implementing the chemical synthesis processes in a homogeneous solution or on a solid phase. In this aspect, reference is made to, e.g., Chiu-Heng Chen et al. (2010), J. Pept. Sci, 16: 231-241.

According to this invention, the isolated peptide and the target substance can be conjugated using chemical, biochemical, enzymatic or genetic coupling methods commonly used by relevant researchers and technicians in the art to which this invention belongs.

In a preferred embodiment of this invention, the isolated peptide is chemically conjugated with the target substance via a chemical crosslinker. The chemical crosslinkers suitable for use in this invention include, but are not limited to, dicyclohexylcarbodiimide (DCC), N-hydroxysuccinimide (NHS), maleimidobenzoyl-N-hydroxysuccinimide ester (MBS), N-isobutyloxy-carbonyl-2-isobutyloxy-1,2-dihydroquinoline (IIDQ).

In a further preferred embodiment of this invention, the target substance is a protein or peptide that forms a fusion protein with the isolated peptide. The fusion protein of this invention can be synthesized by recombinant DNA methods commonly used by relevant researchers and technicians in the art to which this invention belongs. For example, the Examples exemplified the recombinant synthesis of a number of fusion proteins constituted of GFP fused with various isolated peptides according to this invention, in which GFP served as a reporter in the nuclear transport assay.

Accordingly, this invention further provides a nucleic acid construct encoding a fusion protein comprising an isolated peptide as described above and a target protein to be delivered into the nucleus of a mammalian cell, wherein the nucleic acid construct comprises a first nucleic acid fragment encoding the isolated peptide as described above, and a second nucleic acid fragment fused with the first nucleic acid fragment and encoding the target protein.

This invention also provides an expression cassette capable of expressing a fusion protein comprising an isolated peptide of this invention and a target protein to be delivered into the nucleus of a mammalian cell, wherein the expression cassette comprises the nucleic acid construct described above and a promoter operably linked to the nucleic acid construct.

According to this invention, the target protein may be selected from the group consisting of antibodies, antigens, antibacterial peptides, hormones, growth factors, enzymes, and combinations thereof.

Preferably, the promoter is selected from the group consisting of a tac promoter, a CMV promoter, a GAP promoter, a SV40 initial promoter, a RSV-promoter, a HSV-TK promoter, a U6 promoter, a CMV-HSV thymidine kinase promoter, a SRα promoter, and a HIV.LTR promoter. In a preferred embodiment of this invention, the promoter is a CMV promoter.

In a preferred embodiment of this invention, the expression cassette is carried in a vector to form a recombinant vector. Vectors suitable for use in this invention include those commonly used in genetic engineering technology, such as plasmids, cosmids, viruses, or retroviruses.

Vectors suitable for use in this invention may include other expression control elements, such as a transcription starting site, a transcription termination site, a ribosome binding site, a RNA splicing site, a polyadenylation site and a translation termination site, etc. Vectors suitable for use in this invention may further include additional regulatory elements, such as a transcription/translation enhancer sequence, a Shine-Dalgarno sequence, a regulatory sequence and at least a marker gene (e.g., an antibiotic-resistance gene) or a reporter gene allowing for the screening of the vectors under suitable conditions.

According to this invention, any delivery method that could carry DNAs into cells can be used for delivery of the recombinant vector of this invention. For example, the recombinant vector can be introduced into a cell via an approach selected from the group consisting of gene gun or particle bombardment, electroporation, microinjection, heat shock, calcium phosphate precipitation, magnetofection, lipofection, receptor-mediated transfection, viral vector-mediated transfection, use of a transfection reagent, use of a cationic polymer, and any combination thereof.

This invention further provides a method of transporting a target substance into the nucleus of a cell, comprising: contacting the cell with a nuclear transport system comprising the target substance, an isolated peptide as described above and a binding reagent that enables the nuclear transport system to enter into the mammalian cell before the target substance is transported into the nucleus of the mammalian cell, wherein the target substance, the isolated peptide and the binding reagent are associated with each other.

The definitions of the target substance and the binding reagent as described above may apply here.

When the target substance is a target protein, the method of this invention may comprise contacting a mammalian cell with a recombinant vector carrying an expression cassette capable of expressing a fusion protein comprising an isolated peptide as described above and the target protein, wherein the expression cassette comprises a nucleic acid construct comprising a first nucleic acid fragment encoding the isolated peptide as described above, and a second nucleic acid fragment fused with the first nucleic acid fragment and encoding the target protein.

The contacting of the recombinant vector and the mammalian cell may be implemented using any delivery method for DNAs as described above.

In view of the biological activity thereof as disclosed herein, the isolated peptide of this invention is contemplated to have a wide range of use in the fields of medicine, pharmacy, biotechnology, genetic engineering, etc., for the nuclear transport of target substances with known function(s). The NLS peptide of this invention may also be used in exploring or identifying the possible biological activity/function of a novel protein, in developing a method for regulating the gene expression of a sense polynucleotide molecule, or developing a new therapeutic method for a disease using a known compound.

Depending on the function of a target substance that has a known pharmaceutical activity, a conjugate comprising the isolated peptide of this invention and said target substance may be manufactured into a dosage form suitable for parenteral, topical or oral administration using techniques commonly used in the art. A dosage form comprising said conjugate may further include a pharmaceutically acceptable carrier. Selection of an appropriate pharmaceutically acceptable carrier will depend on the sort of the dosage form and the manner of administration of said dosage form, which fall within the routine skill of relevant researchers and technicians in the art to which this invention belongs.

This invention will be further described by way of the following examples. However, it should be understood that the following examples are solely intended for the purpose of illustration and should not be construed as limiting the invention in practice.

### EXAMPLES

### General Experimental Materials

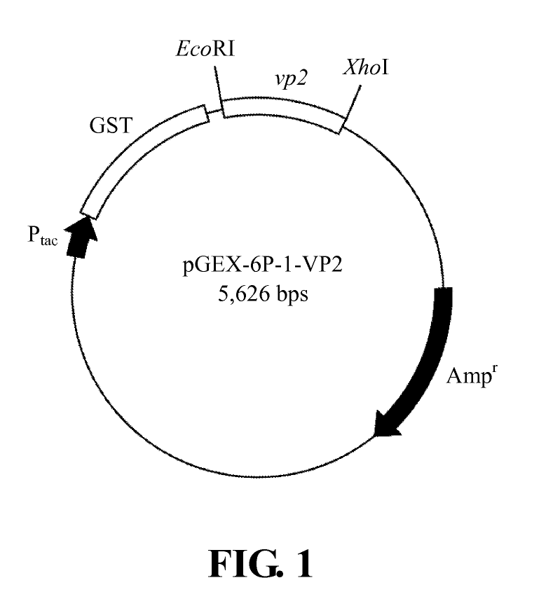

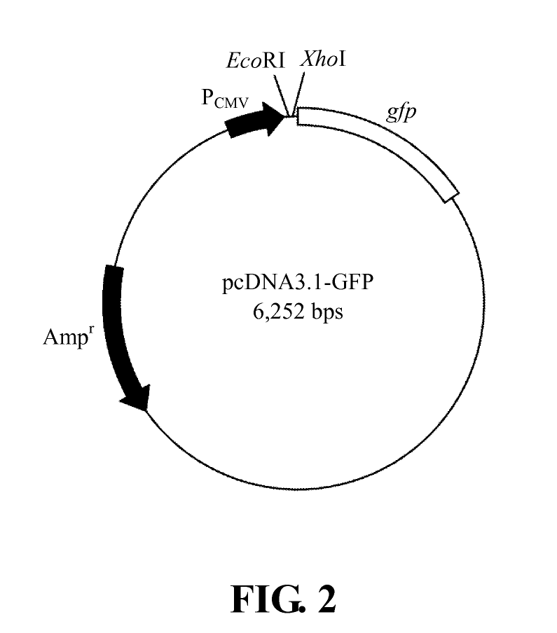

1. Plasmid pGEX-6P-1-VP2 (5,626 bps, see SEQ ID NO: 22 and FIG. 1) was kindly provided by Professor Yi-Yang Lien (Department of Veterinary Medicine, National Pingtung University of Science and Technology, Pingtung County, Taiwan; Meng-Shiou Lee et al. (2009), supra), carrying, amongst others, a tac promoter (P_{tac}), a glutathione S-transferase (GST) encoding gene, an ampicillin-resistance gene (Amp^{r}), an EcoRI recognition site, a XhoI recognition site, and a vp2 gene encoding a VP2 protein of the CAV Taiwan CIA-89 strain, in which the vp2 gene was flanked by the EcoRI and XhoI recognition sites at its 5′- and 3′-terminals, respectively.
2. Plasmid pcDNA3.1-GFP (6,252 bps, see SEQ ID NO: 23 and FIG. 2) was kindly provided by Professor Min-Ying Wang (the Graduate Institute of Biotechnology, National Chung Hsing University, Taichung city, Taiwan), carrying, amongst others, a CMV promoter (P_{CMV}), a gfp gene encoding a green fluorescent protein (GFP), an ampicillin-resistance gene (Amp^{r}), an EcoRI recognition site, and a XhoI recognition site.
3. Primers used in the polymerase chain reaction (PCR) experiments, infra, were synthesized by Genomics Biosci & Tech Co. Ltd. (New Taipei City, Taiwan).
4. The following materials were purchased from Life Technologies, USA: Invitrogen™ Dulbecco's minimal essential medium (DMEM; Cat. No. 11960-069); GIBCO® Dulbecco's Modified Eagle Medium: Nutrient Mixture F-12 (DMEM/F-12) medium (Cat. No. 11320-033); Opti-MEM®I Reduced Serum Medium (Cat. No. 31985); Platinum® Taq DNA polymerase High Fidelity and 10× Platinum® Taq DNA polymerase buffer (Cat. No. 11304-029); GIBCO™ Penicillin-Streptomycin liquid (Cat. No. 15070063) and GIBCO® fetal bovine serum (FBS; Cat. No. 16140-071).
5. The following materials were purchased from QIAGEN: QIAquick PCR Purification Kit (Cat. No. 28106); and QIAGEN Plasmid Mini Kit (Cat. No. 12125).
6. The following materials were purchased from Yeastern Biotech Co., Ltd. (New Taipei City, Taiwan): yT&A® Cloning Vector Kit (Cat. No. YC001); and competent E. coli cells (Cat. No. YE608).
7. The following materials were purchased from Thermo Fisher Scientific Inc., Canada: TurboFect™ in vitro Transfection Reagent (Cat. No. R0531); restriction enzymes FastDigest® EcoRI (Cat. No. FD0274) and FastDigest®XhoI (Cat. No. FD0694); T4 DNA ligase (Cat. No. EL0016); dNTPs (Cat. No. R0181); and MgSO₄ (Cat. No. M2643).
8. The following materials were purchased from Stratagene, USA: PfuUltra™ High-Fidelity DNA Polymerase (Cat. No. 600380); and PfuUltra™ II Fusion HS DNA polymerase (Cat. No. 600670).
9. The following materials were purchased from Sigma-Aldrich Co. LLC., USA: agar (Cat. No. A5306); ampicillin (Cat. No. A0166); LB broth (Cat. No. L3022); phosphate-buffered saline (PBS; Cat. No. P5368); paraformaldehyde (Cat. No. 158127); Triton X-100 (Cat. No. X-100); and 4′,6-diamidino-2-phenylindole (DAPI; Cat. No. D8417).
10. ULTRAhyb®-Oligo hybridization buffer (Cat. No. AM8663) was purchased from Ambion, Inc.
11. HeLa cells (BCRC 60005) and Chinese hamster ovary (CHO) cells (BCRC 60006) used in the transfection experiments, infra, were purchased from the Biosource Collection and Research Center of the Food Industry Research and Development Institute (BCRC of FIRDI, 331 Shih-Pin Road, Hsinchu City 300, Taiwan). HeLa cells were grown in a 75-cm² flask (BD Falcon™; Cat. No. 353136) containing DMEM supplemented with 10% (v/v) FBS, 100 units/mL penicillin, and 100 μg/mL streptomycin. CHO cells were grown in a 75-cm² flask containing GIBCO® DMEM/F-12 medium supplemented with 10% (v/v) FBS, 100 units/mL penicillin, and 100 μg/mL streptomycin. These two cell lines were cultivated in an incubator with culture conditions set at 37° C. and 95% O₂/5% CO₂. Medium changes were performed every four days. Cell passage was performed when the cultured cells reached 80% of confluence.

### General Experimental Procedures:

Concerning the experimental methods and relevant techniques for DNA cloning as employed in this invention, such as DNA cleavage reaction by restriction enzymes, polymerase chain reaction (PCR), DNA ligation with T4 DNA ligase, agarose gel electrophoresis, plasmid transformation, etc., reference is made to a textbook widely known in the art: Sambrook J, Russell DW (2001) Molecular Cloning: a Laboratory Manual, 3rd ed., Cold Spring Harbor Laboratory Press, New York. Site-directed mutagenesis PCR was performed substantially according to the procedures as set forth in L Zheng, et al. (2004) Nucl. Acids Res., 32:e115). These techniques can be readily performed by those skilled in the art based on their professional knowledge and experience.

1. Transformation of E. coli Cells:

A selected plasmid was evenly admixed with competent E. coil cells, followed by standing on ice for 5 minutes (min). The resultant mixture was subsequently spread onto solid agar plates containing 50 μg/mL ampicillin. After cultivation at 37° C. for 16 hours, ampicillin-resistant colonies were picked up from the solid agar plates and then inoculated into LB broth containing 50 μg/mL ampicillin, followed by cultivation at 37° C. for 16 hours.

### 2. Transfection of HeLa Cells and CHO Cells:

A selected plasmid was transfected into either the HeLa cells or the CHO cells using the TurboFect™ in vitro Transfection Reagent according to the manufacturer's instructions. Briefly, 3 μL of the selected plasmid (2 μg/μL, in Opti-MEM®I Reduced Serum Medium) was admixed with 3 μL of the TurboFect™ in vitro Transfection Reagent to form a transfection mixture. In the meantime, each well of 24-well culture plates (BD Falcon™; Cat. No. 353047) was plated with cells for conducting transfection (2×10⁴ cells/500 μL growth medium/well for HeLa cells, and 8×10⁴ cells/500 μL growth medium/well for CHO cells), followed by cultivation in an incubator (37° C., 95% O₂/5% CO₂) for 4 hours. After cell attachment, the liquid in each well of the 24-well plates was removed, and the transfection mixture was subsequently added, followed by incubation for 6 hours. Thereafter, each well of the 24-well plates was replaced with the complete growth medium to a final liquid volume of 1 mL, followed by cultivation of the cells for further 48 hours after transfection.

### 3. Fluorescence Observation on Transfected Cells:

After the transfection treatment described above, cells in each well of the 24-well plates were washed with 500 μL 1×PBS for three times and then treated with 500 μL of a fixation solution (4% paraformaldehyde in 1×PBS) at room temperature for 15 min, followed by washing with 500 μL 1×PBS for three times so as to remove paraformaldehyde. The fixed cells were subsequently incubated with 200 μL of a permeabilization solution (1×PBS containing 0.25% Triton X-100) at 25° C. for 10 min and then washed with 500 μL 1×PBS for three times. Thereafter, the cells were stained in dark with 1 μg/mL 4′,6-diamidino-2-phenylindole (DAPI) in 1×PBS at room temperature for 10 min so as to locate cell nuclei, followed by washing with 500 μL 1×PBS for three times. Finally, the cells were subjected to observation using a Zeiss AxioVert 200 inverted microscope equipped with a 40× objective and an AxioCam HRm CCD camera, in which the GFP fluorescence images and the DAPI images were captured at an excitation wavelength of 480 nm or 350 nm, respectively. The location of GFP is indicated by the emitted green fluorescence in a fluorescence image, and the location of a cell nucleus is indicated by the emitted blue fluorescence in a DAPI image. Image processing was done using Photoshop.

### Example 1

### Subcellular Localization of VP2-GFP Fusion Protein in Mammalian Cells

### Experimental Procedures:

A. Construction of Recombinant Plasmid pcDNA3.1-VP2-GFP:

Based on the nucleotide residues at positions 954-977 and 1,593-1,607 in the nucleotide sequence (SEQ ID NO: 22) of the plasmid pGEX-6P-1-VP2, a VP2 forward primer F1 and a VP2 reverse primer R1 were designed:

```
VP2 forward primer F1
(SEQ ID NO: 24)
5′-tggaattcatgcacgggaacggcgga-3′
          EcoRI
VP2 reverse primer R1
(SEQ ID NO: 25)
5′-tcctcgagcactatacgtaccgg-3′
          XhoI
```

in which the underlined nucleotides represent the recognition site of a restriction enzyme as indicated below.

With the plasmid pGEX-6P-1-VP2 as a template, a first PCR product (664 bps) containing no stop codon and encoding a VP2 protein of the CAV Taiwan CIA-89 strain was obtained from a PCR experiment using the VP2 forward primer F1 and the VP2 reverse primer R1 described above and the PCR reaction conditions shown in Table 1, followed by a 1% agarose gel electrophoresis for molecular weight verification, and recovery and purification using the QIAquick PCR Purification Kit.

TABLE 1
PCR reaction conditions used for the amplification
of the first PCR product

| Contents | Volume (μL) |
|---|---|
| pGEX-6P-1-VP2 (0.1 μg/μL) | 1 |
| VP2 forward primer F1 (12.5 μM) | 2 |
| VP2 reverse primer R1 (12.5 μM) | 2 |
| dNTPs (2.5 mM) | 4 |
| Platinum ® Taq DNA polymerase buffer (10 X) | 5 |
| Platinum ® Taq DNA polymerase High Fidelity (5 U/μL) | 1 |
| MgSO₄ (50 mM) | 2 |
| ddH₂O | 33 |

Operation conditions: Denaturation at 95° C. for 5 min, followed by 30 cycles of the following reactions: denaturation at 95° C. for 60 sec, primer annealing at 55° C. for 60 sec, and extension at 72° C. for 2 min; and finally elongation at 72° C. for 5 min.

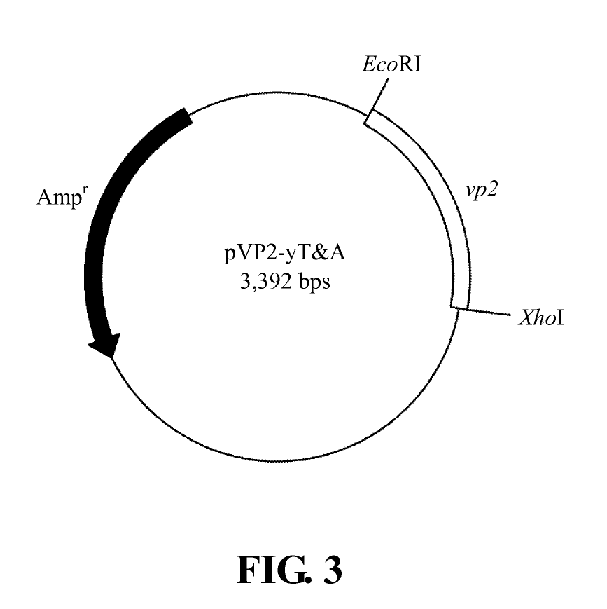

A recombinant plasmid pVP2-yT&A (3,392 bps) that contained the first PCR product and had a structure as shown in FIG. 3 was subsequently obtained using the yT&A® Cloning Vector Kit, followed by transformation using competent E. coli cells according to the procedures as described in the General Experimental Procedures, and extraction using the QIAGEN Plasmid Mini Kit. According to the sequencing analysis conducted by Genomics Biosci & Tech Co. Ltd., a vp2 gene having a nucleotide sequence as shown in SEQ ID NO: 3 was included in the recombinant plasmid pVP2-yT&A.

The recombinant plasmid pVP2-yT&A was cleaved with restriction enzymes EcoRI and XhoI so that a first cleavage product (654 bps) containing the vp2 gene of SEQ ID NO: 3 was obtained. In the meantime, plasmid pcDNA3.1-GFP was cleaved with restriction enzymes EcoRI and XhoI, so that a second cleavage product (6,219 bps) containing the gfp gene was obtained. The first and second cleavage products were then mixed at a molar ratio of 3:1 and ligated using T4 DNA ligase. The ligated product thus obtained was subsequently transformed into competent E. coil cells according to the procedures as described in the General Experimental Procedures, followed by extraction using the QIAGEN Plasmid Mini Kit.

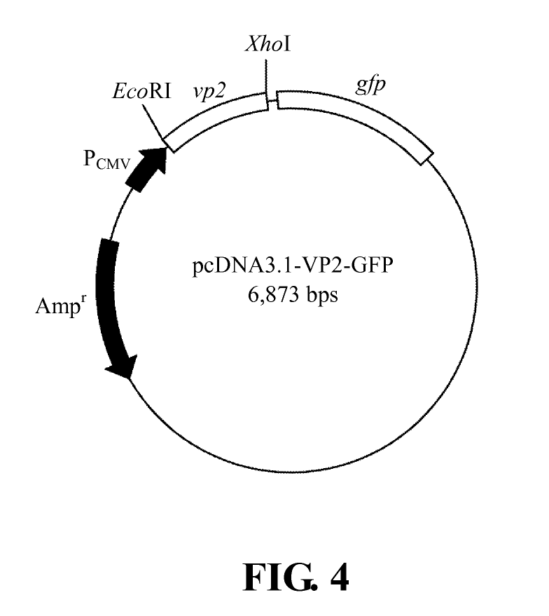

An E. coli transformant thus obtained was verified to harbor a recombinant plasmid named “pcDNA3.1-VP2-GFP,” which was determined to have a plasmid construct as shown in FIG. 4, in which the vp2 gene of SEQ ID NO: 3 was fused with and located upstream of the gfp gene, so that a VP2-GFP fusion protein could be expressed. As such, the subcellular localization of said VP2-GFP fusion protein can be verified by observing the green fluorescence generated by GFP.

### B. Localization of VP2-GFP Fusion Protein in Mammalian Cells:

The recombinant plasmid pcDNA3.1-VP2-GFP as obtained in the preceding section A was transfected into HeLa cells or CHO cells according to the procedures as described in the General Experimental Procedures, and the transfected cells thus obtained were subjected to fluorescence observation according to the procedures as described in the General Experimental Procedures. For comparison, the same experiments were performed using the plasmid pcDNA3.1-GFP as a control.

### Results:

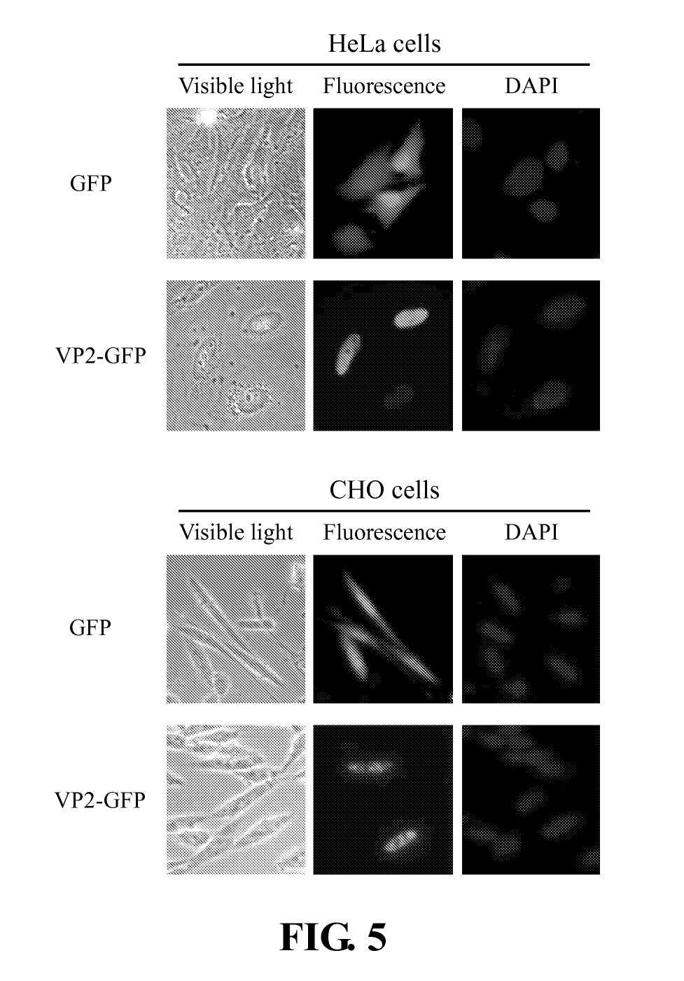

FIG. 5 shows the expression of GFP or VP2-GFP in HeLa cells (upper part) or CHO cells (lower part) after transfection with a control plasmid pcDNA3.1-GFP or the recombinant plasmid pcDNA3.1-VP2-GFP, as observed by a Zeiss AxioVert 200 inverted microscope under 400× magnification. Referring to FIG. 5, for cells transfected with plasmid pcDNA3.1-GFP, emitted green fluorescence was observed in the nucleus and cytoplasm areas. In contrast, for cells transfected with plasmid pcDNA3.1-VP2-GFP, emitted green fluorescence was observed in the nucleus areas only. The experimental results indicated that the VP2-GFP fusion protein exhibited a nuclear localization signal (NLS) function, leading the applicants to presume that the VP2 protein might include therein a functional NLS peptide that directs the nuclear transport of a target substance, in particular a protein of interest, into the nucleus of a cell.

### Example 2

### Sequence Alignment of VP2 Proteins from Different Isolated Strains of CAV and Prediction of NLS Peptide in CAV VP2 Protein

In this example, the applicants applied an in silico method to predict the existence and location of NLS peptide(s) in each of the VP2 proteins of different isolated strains of CAV.

The VP2 protein of the CAV Taiwan CIA-89 strain has an amino acid sequence as shown in SEQ ID NO: 8 (Meng-Shiou Lee et al. (2009), supra). The applicants compared the amino acid sequence of the VP2 protein of the CAV Taiwan CIA-89 strain with those of the VP2 proteins of a number of isolated strains of CAV as deposited in the UniProtKB database, including:

Australia/CAU269-7/2000 (UniProtKB Accession Number: Q9IZU7),

Germany Cuxhaven-1 (UniProtKB Accession Number: P69484),

Japan 82-2 (UniProtKB Accession Number: P54093),

USA 26p4 (UniProtKB Accession Number: P54092), and

USA CIA-1 (UniProtKB Accession Number: P69485).

The sequence divergence amongst the VP2 proteins of these six isolated strains of CAV was analyzed using the Biology Workbench 3.2 software.

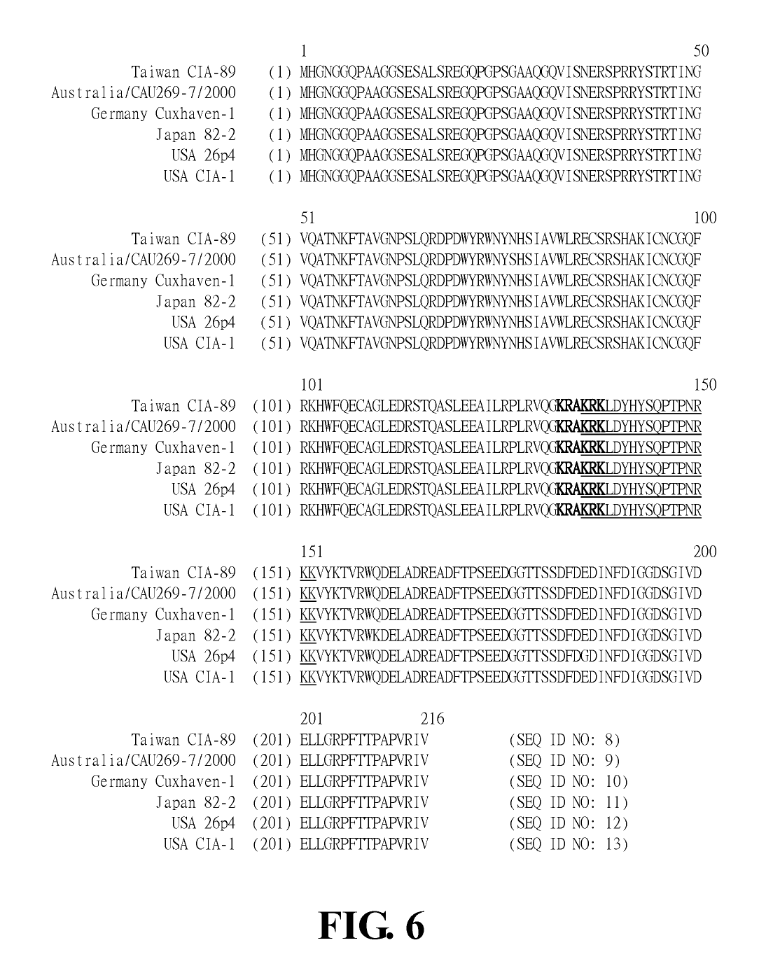

The sequence alignment results thus obtained are shown in FIG. 6, which reveals that the amino acid sequence of the CAV VP2 protein is highly conserved in different isolated strains. Therefore, in order to explore NLS peptide(s), the full-length amino acid sequence of the VP2 protein of the CAV Taiwan CIA-89 strain was further used and examined using the WoLF PSORT and NLStradamus softwares.

A BiNLS1 motif (SEQ ID NO: 4) was predicted by the WoLF PSORT software, with a putative motif position spanning amino acid residues 136-152 of the CAV VP2 protein (see the underlined amino acid residues shown in FIG. 6). On the other hand, a NLS2 motif (SEQ ID NO: 5) was predicted by the NLStradamus software at a prediction cutoff value of 0.5 and this motif was located at a region spanning amino acid residues 133-138 of the CAV VP2 protein (see the boldfaced amino acid residues shown in FIG. 6). Based on the bioinformatics analysis results, the applicants presumed that the functional NLS peptide(s) in the CAV VP2 protein might be BiNLS1 and/or NLS2.

### Example 3

### Subcellular Localization of Various Truncated VP2-GFP Fusion Proteins in Mammalian Cells

In this example, the applicants constructed a series of recombinant plasmids, each carrying a different truncated vp2-gfp fusion gene encoding a C-terminal or N-terminal truncated VP2-GFP fusion protein (i.e., a C-terminal or N-terminal truncated VP2 protein fused with GFP at the C-terminal). These recombinant plasmids were subsequently transfected into mammalian cells. Based on the observed subcellular localization of the truncated VP2-GFP fusion proteins expressed in the transfected mammalian cells, the possible functional NLS peptide(s) in the CAV VP2 protein was identified.

### Experimental Procedures:

Based on the amino acid sequence of the VP2 protein of the CAV Taiwan CIA-89 strain as shown in SEQ ID NO: 8, the applicants designed three C-terminal truncated VP2 proteins, namely VP2-115dC, VP2-132dC and VP2-145dC, and three N-terminal truncated VP2 proteins, namely VP2-111dN, VP2-141dN and VP2-160dN. In order to clone the corresponding truncated vp2 genes encoding these six truncated VP2 proteins, six primer pairs as shown in Table 2 were designed based on the vp2 gene of the CAV Taiwan CIA-89 strain carried in the plasmid pGEX-6P-1-VP2, said vp2 gene corresponding to nucleotide residues 960 to 1610 in SEQ ID NO: 22.

TABLE 2
The primer pairs designed to clone the various truncated vp2 gene by PCR

| | VP2 protein's | | | | |
|---|---|---|---|---|---|
| | amino acid | | | The correspond- | |
| | residues en- | | | ing nucleotide | The size |
| | coded by the | | | residues in | of PCR |
| Truncated | truncated | | The primer's nucleotide | the plasmid | product |
| vp2 gene | vp2 gene | Primer | sequence (5′→3′) | pGEX-6P-1-VP2 | (bp) |
| vp2-115dC | 1-115 | VP2 forward | tggaattcatgcacgggaacggcgga | 960-977 | 361 |
| | | primer F1 | (SEQ ID NO: 24) | | |
| | | | EcoRI | | |
| | | VP2 reverse | tcctcgagtgatcggtcctcaagt | 1304-1289 | |
| | | primer R2 | (SEQ ID NO: 26) | | |
| | | | XhoI | | |
| vp2-132dC | 1-132 | VP2 forward | tggaattcatgcacgggaacggcgga | 960-977 | 412 |
| | | primer F1 | (SEQ ID NO: 24) | | |
| | | | EcoRI | | |
| | | VP2 reverse | tcctcgagaccctgtactcggag | 1355-1341 | |
| | | primer R3 | (SEQ ID NO: 27) | | |
| | | | XhoI | | |
| vp2-145dC | 1-145 | VP2 forward | tggaattcatgcacgggaacggcgga | 960-977 | 451 |
| | | primer F1 | (SEQ ID NO: 24) | | |
| | | | EcoRI | | |
| | | VP2 reverse | tcctcgagctgggagtagtggtaatc | 1394-1377 | |
| | | primer R4 | (SEQ ID NO: 28) | | |
| | | | XhoI | | |
| vp2-111dN | 112-216 | VP2 forward | tggaattcatggaggaccgatcaacc | 1293-1307 | 334 |
| | | primer F2 | (SEQ ID NO: 29) | | |
| | | | EcoRI | | |
| | | VP2 reverse | tcctcgagcactatacgtaccgg | 1607-1593 | |
| | | primer R1 | (SEQ ID NO: 25) | | |
| | | | XhoI | | |
| vp2-141dN | 142-216 | VP2 forward | aggaattcatgcactactcccagccg | 1383-1397 | 244 |
| | | primer F3 | (SEQ ID NO: 30) | | |
| | | | EcoRI | | |
| | | VP2 reverse | tcctcgagcactatacgtaccgg | 1607-1593 | |
| | | primer R1 | (SEQ ID NO: 25) | | |
| | | | XhoI | | |
| vp2-160dN | 161-216 | VP2 forward | aggaattcatggacgagctcgcagac | 1440-1454 | 187 |
| | | primer F4 | (SEQ ID NO: 31) | | |
| | | | EcoRI | | |
| | | VP2 reverse | tcctcgagcactatacgtaccgg | 1607-1593 | |
| | | primer R1 | (SEQ ID NO: 25) | | |
| | | | XhoI | | |

Note:
The underlined nucleotides represent the recognition site of a restriction enzyme as indicated below.

With the plasmid pGEX-6P-1-VP2 as a template, six different PCR products, each having a size as expected and containing a desired truncated vp2 gene as shown in Table 2, were obtained using the corresponding primer pairs listed in Table 2 and the PCR reaction conditions as shown in Table 1, except that in the 30 cycles of reactions, denaturation was conducted at 95° C. for 45 sec and primer annealing was conducted at 55° C. for 45 sec, followed by a 2% agarose gel electrophoresis for molecular weight verification, and recovery and purification using the QIAquick PCR Purification Kit.

Six different recombinant plasmids, each respectively containing one of the aforesaid six PCR products, were subsequently obtained using the yT&A® Cloning Vector Kit, followed by transformation using competent E. coli cells according to the procedures as described in the General Experimental Procedures, and extraction using the QIAGEN Plasmid Mini Kit. According to the sequencing analysis conducted by Genomics Biosci & Tech Co. Ltd., these six recombinant plasmids, which were named pVP2-115dC-yT&A (3,089 bps), pVP2-132dC-yT&A (3,140 bps), pVP2-145dC-yT&A (3,179 bps), pVP2-111dN-yT&A (3,062 bps), pVP2-141dN-yT&A (2,972 bps) and pVP2-160dN-yT&A (2,915 bps), respectively, were confirmed to carry the corresponding truncated vp2 genes as indicated in Table 2.

The six recombinant plasmids as obtained above were separately used to construct a recombinant plasmid carrying a truncated vp2-gfp fusion gene substantially according to the procedures as set forth in Example 1 for the construction of recombinant plasmid pcDNA3.1-VP2-GFP.

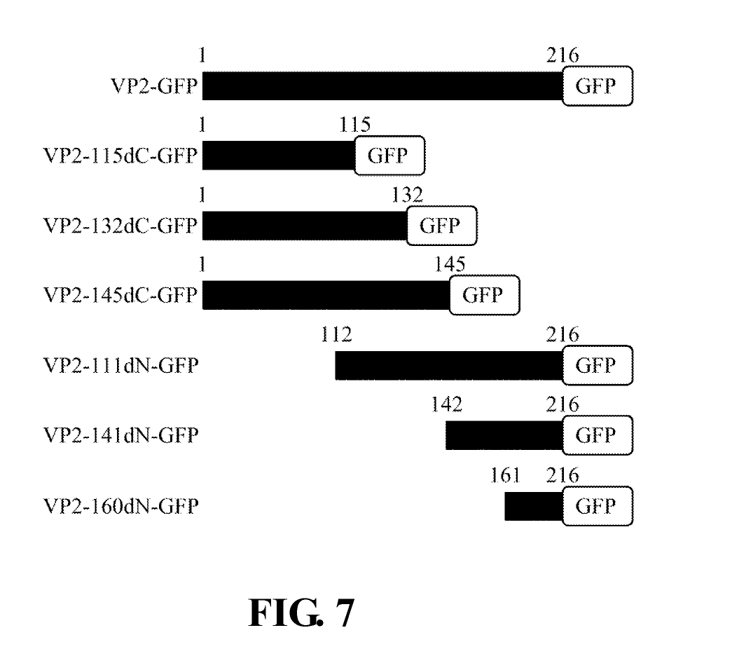

Six recombinant plasmids, each carrying a truncated vp2-gfp fusion gene encoding a corresponding truncated VP2-GFP fusion protein as shown in FIG. 7, were obtained and named as pcDNA3.1-VP2-115dC-GFP (6,570 bps), pcDNA3.1-VP2-132dC-GFP (6,621 bps), pcDNA3.1-VP2-145dC-GFP (6,660 bps), pcDNA3.1-VP2-111dN-GFP (6,543 bps), pcDNA3.1-VP2-141dN-GFP (6,453 bps) and pcDNA3.1-VP2-160dN-GFP (6,396 bps), respectively. These six recombinant plasmids were subsequently transfected into HeLa cells or CHO cells according to the procedures as described in the General Experimental Procedures, and the transfected cells thus obtained were subjected to fluorescence observation according to the procedures as described in the General Experimental Procedures.

### Results:

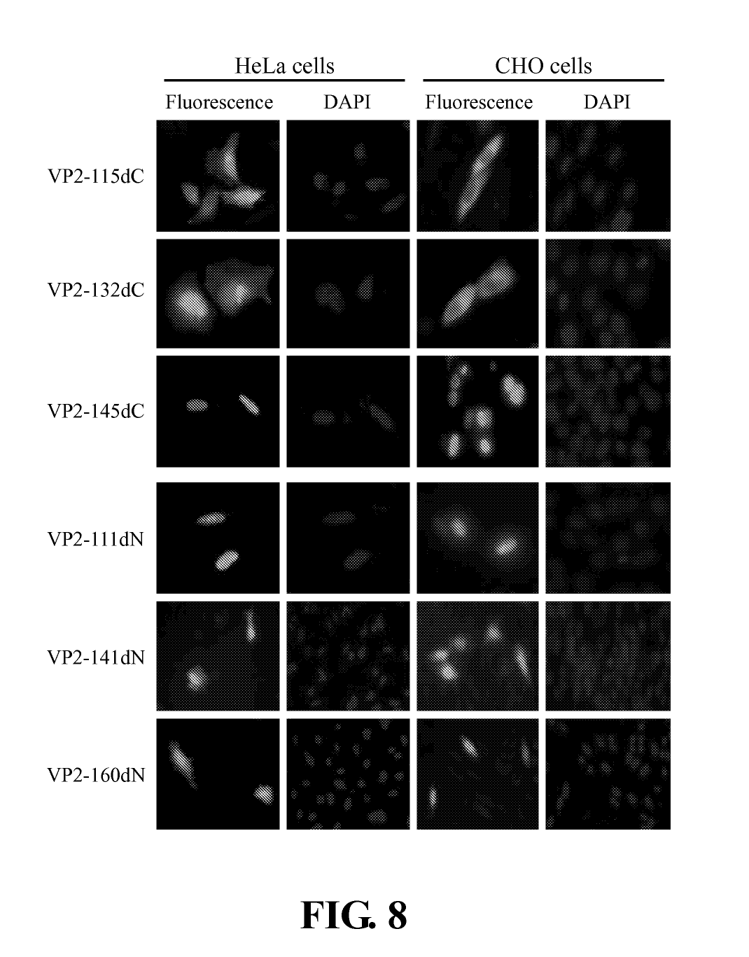

FIG. 8 shows the microscopic examination results of HeLa cells and CHO cells after transfection with plasmids pcDNA3.1-VP2-115dC-GFP (represented by “VP2-115dC”), pcDNA3.1-VP2-132dC-GFP (represented by “VP2-132dC”), pcDNA3.1-VP2-145dC-GFP (represented by “VP2-145dC”), pcDNA3.1-VP2-111dN-GFP (represented by “VP2-111dN”), pcDNA3.1-VP2-141dN-GFP (represented by “VP2-141dN”) and pcDNA3.1-VP2-160dN-GFP (represented by “VP2-160dN”), as observed by a Zeiss AxioVert 200 inverted microscope under 400× magnification. According to the results shown in FIG. 8, the subcellular localization of various truncated VP2-GFP fusion proteins in mammalian cells transfected by the aforesaid six plasmids are summarized in Table 3.

TABLE 3
Subcellular localization of various truncated
VP2-GFP fusion proteins in mammalian cells

Subcellular localization

| Truncated VP2-GFP fusion protein | HeLa cells | CHO cells |
|---|---|---|
| VP2-115dC-GFP | N/C | C |
| VP2-132dC-GFP | N/C | C |
| VP2-145dC-GFP | N | N |
| VP2-111dN-GFP | N | N |
| VP2-141dN-GFP | N/C | N/C |
| VP2-160dN-GFP | N/C | N/C |

Note:
N represents nucleus, C represents cytoplasm, and N/C represents nucleus and cytoplasm.

It can be seen from FIG. 8 and Table 3 that densely emitted green fluorescence was observed in the nucleus areas of cells transfected with plasmid pcDNA3.1-VP2-145dC-GFP or pcDNA3.1-VP2-111dN-GFP, whereas evenly distributed green fluorescence was observed in the cytoplasm areas of cells transfected with the other four plasmids. The obtained results indicated that an intact NSL peptide was present in the truncated VP2-145dC-GFP protein, which contained a C-terminal truncated VP2 protein having an amino acid sequence as shown in SEQ ID NO: 20 (corresponding to amino acid residues 1-145 of the VP2 protein of SEQ ID NO: 8), and in the truncated VP2-111dN-GFP protein, which contained a N-terminal truncated VP2 protein having an amino acid sequence as shown in SEQ ID NO: 21 (corresponding to amino acid residues 112-216 of the VP2 protein of SEQ ID NO: 8). Based on this finding, it was presumed that a NLS peptide might be located at a region spanning amino acid residues 112-145 of the CAV VP2 protein.

According to the sequence comparison results, the VP2-145dC-GFP fusion protein is longer by 13 amino acid residues (corresponding to amino acid residues 133-145 of the VP2 protein of SEQ ID NO: 8) than the VP2-132dC-GFP fusion protein at the C-terminal, and the VP2-111dN-GFP fusion protein is longer by 30 amino acid residues (corresponding to amino acid residues 112-141 of the VP2 protein of SEQ ID NO: 8) than the VP2-141dN-GFP fusion protein at the N-terminal. The sequence divergence influences the nuclear localization abilities of these truncated VP2-GFP fusion proteins. It was therefore presumed that a NLS peptide of SEQ ID NO: 6 might be located at a region spanning amino acid residues 133-141 of the CAV VP2 protein. Specifically, the NLS2 motif (corresponding to amino acid residues 133-138 of the VP2 protein of SEQ ID NO: 8) as predicted in Example 2 was fully covered by the region of amino acid residues 133-141. In contrast, the BiNLS1 motif (corresponding to amino acid residues 136-152 of the VP2 protein of SEQ ID NO: 8) as predicted in Example 2 was partially covered by the region of amino acid residues 133-141. The applicants thus presumed that the NLS peptide of the CAV VP2 protein might be the NLS2 motif as predicted.

### Example 4

### Subcellular Localization of Various Mutant VP2-GFP Fusion Proteins in Mammalian Cells

In order to identify which putative motif(s) predicted in Example 2, i.e., BiNLS1 and/or NLS2, played a role in the nuclear localization ability of the CAV VP2 protein, in this example, various site-directed mutations were introduced into the amino acid sequence of the CAV VP2 protein at positions 133-134, 136-138 and 150-152 where basic amino acids were located, so as to evaluate the criticality of these basic amino acid residues to the CAV VP2 protein in terms of nuclear localization ability.

### Experimental Procedures:

In order to clone a series of mutant vp2-gfp genes encoding various mutant VP2-GFP fusion proteins, each being constituted of a desired mutant VP2 protein fused with a GFP protein, eight primer pairs as shown in Table 4 were designed based on the vp2 gene of SEQ ID NO: 3 as carried in plasmid pcDNA3.1-VP2-GFP obtained in Example 1, said vp2 gene being located at nucleotide residues 949-1,596 of plasmid pcDNA3.1-VP2-GFP. The amino acid mutations introduced in the mutant VP2 proteins and the nucleotide positions in plasmid pcDNA3.1-VP2-GFP that correspond to each one of the designed primers are also indicated in Table 4.

TABLE 4
Primer pairs designed to introduce site-directed mutations into the vp2 gene by PCR

| Primer | Mutant VP2 | Mutation | | The primer's nucleotide | Nucleotide |
|---|---|---|---|---|---|
| pair | protein | sites | Primer | sequence (5′→3′) | positions |
| 1 | VP2-136-138A | K136A | Sense | aaacgagctgctgctgctcttgattac | 1345-1371 |
| | | R137A | primer MF1 | (SEQ ID NO: 32) | |
| | | K138A | Anti-sense | gtaatcaagagcagcagcagctcgttt | 1371-1345 |
| | | | primer MR1 | (SEQ ID NO: 33) | |
| 2 | VP2-150-152A | R150A | Sense | accccgaacgcagcagcagtgtataagactgtaagatgg | 1387-1425 |
| | | K151A | primer MF2 | (SEQ ID NO: 34) | |
| | | K152A | Anti-sense | ccatcttacagtcttatacactgctgctgcgttcggggt | 1425-1387 |
| | | | primer MR2 | (SEQ ID NO: 35) | |
| 3 | VP2-136- | R134A | Sense | gtacagggtaaagctgctgctgctgct | 1336-1362 |
| | 138A/134A | K136A | primer MF3 | (SEQ ID NO: 36) | |
| | | R137A | Anti-sense | agcagcagcagcagctttaccctgtac | 1362-1336 |
| | | K138A | primer MR3 | (SEQ ID NO: 37) | |
| 4 | VP2-136- | K133A | Sense | gtacagggtgctcgagctgctgctgct | 1336-1362 |
| | 138A/133A | K136A | primer MF4 | (SEQ ID NO: 38) | |
| | | R137A | Anti-sense | agcagcagcagctcgagcaccctgtac | 1362-1336 |
| | | K138A | primer MR4 | (SEQ ID NO: 39) | |
| 5 | VP2-136-138A/ | K133A | Sense | gtacagggtgctgctgctgctgctgct | 1336-1362 |
| | 133A/134A | R134A | primer MF5 | (SEQ ID NO: 40) | |
| | | K136A | Anti-sense | agcagcagcagcagcagcaccctgtac | 1362-1336 |
| | | R137A | primer MR5 | (SEQ ID NO: 41) | |
| | | K138A | | | |
| 6 | VP2-133A | K133A | Sense | gtacagggtgctcgagctaaaagaaagc | 1336-1363 |
| | | | primer MF6 | (SEQ ID NO: 42) | |
| | | | Anti-sense | gctttcttttagctcgagcaccctgtac | 1363-1336 |
| | | | primer MR6 | (SEQ ID NO: 43) | |
| 7 | VP2-134A | R134A | Sense | gtacagggtaaagctgctaaaagaaagc | 1336-1363 |
| | | | primer MF7 | (SEQ ID NO: 44) | |
| | | | Anti-sense | gctttcttttagcagctttaccctgtac | 1363-1336 |
| | | | primer MR7 | (SEQ ID NO: 45) | |
| 8 | VP2- | K133A | Sense | gtacagggtgctgctgctaaaagaaagc | 1336-1363 |
| | 133A/134A | R134A | primer MF8 | (SEQ ID NO: 46) | |
| | | | Anti-sense | gctttcttttagcagcagcacctgtac | 1363-1336 |
| | | | primer MR8 | (SEQ ID NO: 47) | |

Note:
Each underlined region in the nucleotide sequence of an indicted primer was designed to introduce alanine residue(s) at the mutation site(s) as indicated.

With the plasmid pcDNA3.1-VP2-GFP as a template, five recombinant plasmids, which were later named pcDNA3.1-VP2-136-138A-GFP, pcDNA3.1-VP2-150-152A-GFP, pcDNA3.1-VP2-133A-GFP, pcDNA3.1-VP2-134A-GFP and pcDNA3.1-VP2-133A/134A-GFP, were obtained using the 1st, 2nd, 6th, 7th and 8th primer pairs shown in Table 4 and the PCR reaction conditions as shown in Table 5. The five recombinant plasmids thus obtained were subsequently transformed into competent E. coli cells according to the procedures as described in the General Experimental Procedures, followed by extraction using the QIAGEN Plasmid Mini Kit. According to the sequencing analysis conducted by Genomics Biosci & Tech Co. Ltd., each of these five recombinant plasmids was confirmed to carry a mutant vp2-gfp fusion gene encoding a mutant VP2-GFP fusion protein, in which the fusion protein contained a mutant VP2 protein that was “VP2-136-138A” for the plasmid pcDNA3.1-VP2-136-138A-GFP, “VP2-150-152A” for the plasmid pcDNA3.1-VP2-150-152A-GFP, “VP2-133A” for the plasmid pcDNA3.1-VP2-133A-GFP, “VP2-134A” for the plasmid pcDNA3.1-VP2-134A-GFP, and “VP2-133A/134A” for the plasmid pcDNA3.1-VP2-133A/134A-GFP.

TABLE 5
Reaction conditions for the site-directed
mutagenesis of VP2-encoding genes by PCR

| Contents | Volume (μL) |
|---|---|
| A selected plasmid template (0.1 μg/μL) | 1 |
| A selected sense primer (12.5 μM) | 2 |
| A selected anti-sense primer (12.5 μM) | 2 |
| dNTPs (2.5 mM) | 4 |
| PfuUltra ™ DNA polymerase buffer (10 X) | 5 |
| PfuUltra ™ High Fidelity DNA polymerase (5 U/μL) | 1 |
| ddH₂O | 35 |

Operation conditions: Denaturation at 95° C. for 5 min, followed by 19 cycles of the following reactions: denaturation at 95° C. for 60 sec, primer annealing at 55° C. for 60 sec, and extension at 72° C. for 4 min; and finally elongation at 72° C. for 5 min.

With the plasmid pcDNA3.1-VP2-136-138A-GFP as a template, four additional recombinant plasmids, which were later named pcDNA3.1-VP2-136-138A/150-152A-GFP, pcDNA3.1-VP2-136-138A/134A-GFP, pcDNA3.1-VP2-136-138A/133A-GFP and pcDNA3.1-VP2-136-138A/133A/134A-GFP, were obtained using the 2nd, 3rd, 4th and 5th primer pairs shown in Table 4 and the PCR reaction conditions as shown in Table 5, respectively. The four recombinant plasmids thus obtained were subsequently transformed into competent E. coli cells according to the procedures as described in the General Experimental Procedures, followed by extraction using the QIAGEN Plasmid Mini Kit. According to the sequencing analysis conducted by Genomics Biosci & Tech Co. Ltd., each of these four recombinant plasmids was confirmed to carry a mutant vp2-gfp fusion gene encoding a mutant VP2-GFP fusion protein, in which the fusion protein contained a mutant VP2 protein that was “VP2-136-138A/150-152A” for the plasmid pcDNA3.1-VP2-136-138A/150-152A-GFP, “VP2-136-138A/133A” for the plasmid pcDNA3.1-VP2-136-138A/133A-GFP, “VP2-136-138A/134A” for the plasmid pcDNA3.1-VP2-136-138A/134A-GFP, and “VP2-136-138A/133A/134A” for the plasmid pcDNA3.1-VP2-136-138A/133A/134A-GFP.

The nine recombinant plasmids as obtained above were subsequently transfected into mammalian cells (HeLa cells or CHO cells) according to the procedures as described in the General Experimental Procedures, respectively, and the transfected cells thus obtained were subjected to fluorescence observation according to the procedures as described in the General Experimental Procedures.

### Results:

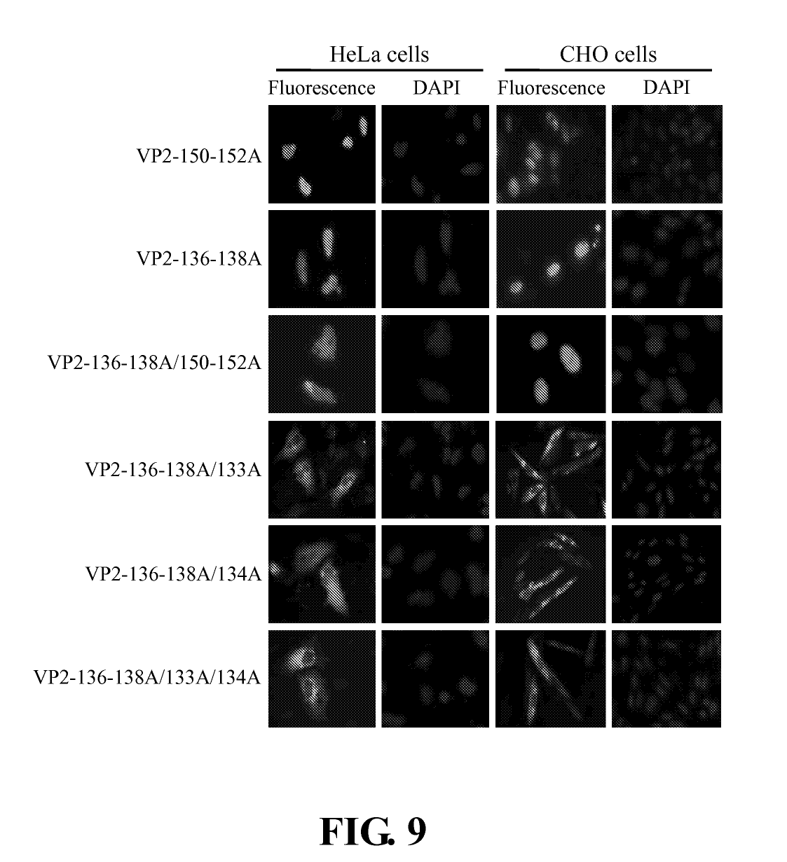

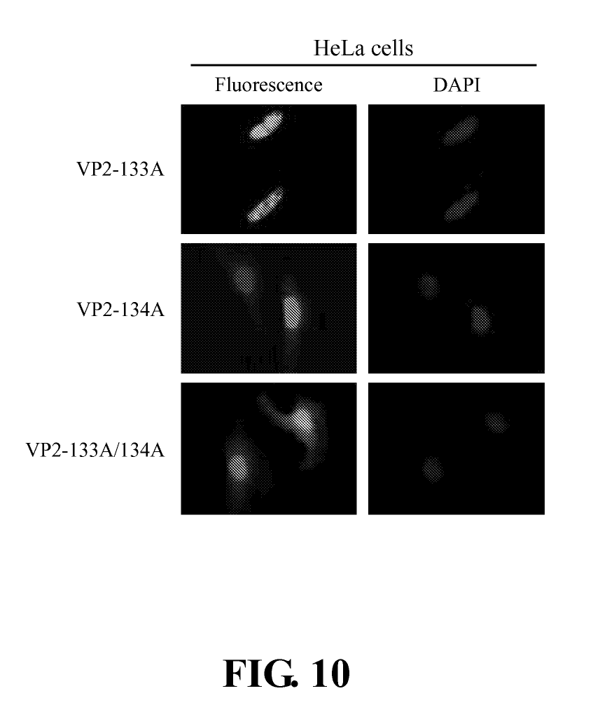

FIG. 9 shows the microscopic examination results of HeLa cells and CHO cells after transfection with plasmids pcDNA3.1-VP2-150-152A-GFP (represented by “VP2-150-152A”), pcDNA3.1-VP2-136-138A-GFP (represented by “VP2-136-138A”), pcDNA3.1-VP2-136-138A/150-152A-GFP (represented by “VP2-136-138A/150-152A”), pcNA3.1-VP2-136-138A/133A-GFP (represented by “VP2-136-138A/133A”), pcDNA3.1-VP2-136-138A/134A-GFP (represented by “VP2-136-138A/134A”) and pcDNA3.1-VP2-136-138A/133A/134A-GFP (represented by “VP2-136-138A/133A/134A”), and FIG. 10 shows the microscopic examination results of HeLa cells after transfection with plasmids pcDNA3.1-VP2-133A-GFP (represented by “VP2-133A”), pcDNA3.1-VP2-134A-GFP (represented by “VP2-134A”) and pcDNA3.1-VP2-133A/134A-GFP (represented by “VP2-133A/134A”), as observed by a Zeiss AxioVert 200 inverted microscope under 400× magnification. According to the results shown in FIGS. 9 and 10, the subcellular localization of various mutant VP2-GFP fusion proteins in mammalian cells transfected by the aforesaid nine plasmids are summarized in Table 6, which also shows the mutation sites in amino acid residues 133-152 of each corresponding mutant VP2 protein, as well as the respective locations of the two predicted BiNLS1 and NLS2 motifs.

TABLE 6
The subcellular localization of various mutant VP2-GFP fusion proteins in
mammalian cells and the mutation sites in amino acid residues 133-152
of each corresponding mutant VP2 protein.

| | Mutation sites in amino acid residues | |
|---|---|---|
| Mutant VP2-GFP | 133-152 of the corresponding mutant | Subcellular |
| fusion protein | VP2 protein | localization |
| VP2-150-152A-GFP | 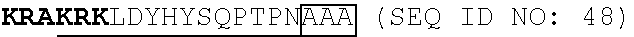 | Nucleus |
| VP2-136-138A-GFP | 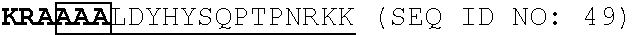 | Nucleus |
| VP2-136-138A/150-152A-GFP | 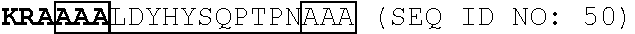 | Nucleus |
| VP2-136-138A/133A-GFP | 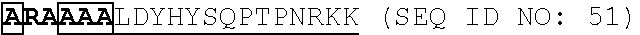 | Cytoplasm |
| VP2-136-138A/134A-GFP | 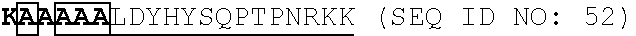 | Cytoplasm |
| VP2-136-138A/133A/134A-GFP | 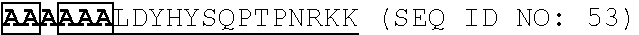 | Cytoplasm |
| VP2-133A-GFP | 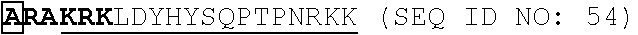 | Nucleus |
| VP2-134A-GFP | 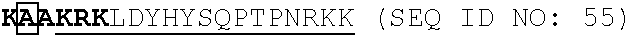 | Nucleus |
| VP2-133A/134A-GFP | 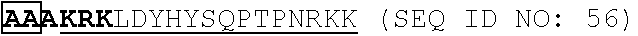 | Nucleus |

Note: The mutation sites are framed; and the locations of the predicted BiNLS1 and NLS2 motifs are underlined and boldfaced, respectively.

It can be seen from FIGS. 9 and 10 as well as Table 6 that densely emitted green fluorescence was observed in the nucleus areas of cells transfected with plasmid pcDNA3.1-VP2-150-152A-GFP, pcDNA3.1-VP2-136-138A-GFP, pcDNA3.1-VP2-136-138A/150-152A-GFP, pcDNA3.1-VP2-133A-GFP, pcDNA3.1-VP2-134A-GFP or pcDNA3.1-VP2-133A/134A-GFP, and evenly distributed green fluorescence was observed in the cytoplasm areas of cells transfected with plasmid pcDNA3.1-VP2-136-138A/133A-GFP, pcDNA3.1-VP2-136-138A/134A-GFP or pcDNA3.1-VP2-136-138A/133A/134A-GFP. The obtained results reveal that mutant VP2 proteins VP2-150-152A (SEQ ID NO: 14), VP2-136-138A (SEQ ID NO: 15), VP2-136-138A′150-152A (SEQ ID NO: 16), VP2-133A (SEQ ID NO: 17), VP2-134A (SEQ ID NO: 18) and VP2-133A/134A (SEQ ID NO: 19), although each having site-directed mutations as shown in Table 4, exhibited nuclear localization abilities comparable to that of the CAV VP2 protein of SEQ ID NO: 8. Inasmuch as the nuclear localization ability of the CAV VP2-protein was not destroyed by site-directed mutations at either amino acid positions 150-152, or amino acid positions 136-138, or both, it was concluded that the BiNLS1 motif as predicted in Example 2 was not the functional NLS peptide contained in the CAV VP2 protein.

According to the results of other mutant VP2 proteins having one or more site-directed mutations at amino acid positions 133, 134 and 136-138, as well as those obtained in Examples 2 and 3, it was further concluded that a functional NLS peptide should be located at a region spanning amino acid residues 133 to 138 of the CAV VP2 protein, which region was matched with the location of the NLS2 motif of SEQ ID NO: 5 as predicted in Example 2. Based on the results summarized in Table 6, a peptide of SEQ ID NO: 57, which was an Ala mutant form of the NLS2 motif of SEQ ID NO: 5, was presumed to be functional in exhibiting the nuclear localization ability as desired.

### Example 5

### Evaluation of the Nuclear Localization Ability of NLS Peptides Derived from the CAV VP2 Protein

According to the experimental results obtained in Examples 3 and 4, in this example, the applicants constructed two VP2 NLS peptides derived from the CAV VP2 protein, namely VP2 (133-138) and VP2 (112-145). VP2 (133-138) was constituted of amino acid residues shown in SEQ ID NO: 5 (i.e., the predicted NLS2 motif in full length) and corresponding to those in positions 133-138 of the CAV VP2 protein of SEQ ID NO: 8. VP2 (112-145), which covered the full-length NLS2 motif, was constituted of amino acid residues shown in SEQ ID NO: 7 and corresponding to those in positions 112-145 of the CAV VP2 protein of SEQ ID NO: 8. These two VP2 NLS peptides were subjected to a nuclear transport assay using the GFP protein as a reporter, so as to evaluate the nuclear localization abilities of NLS peptides derived from the CAV VP2 protein.

### Experimental Procedures:

A. Construction of Recombinant Plasmid pVP2 (112-145)-yT&A carrying a VP2 (112-145)-Encoding Sequence

In order to clone a nucleotide sequence encoding VP2 (112-145) of SEQ ID NO: 7, a VP2 forward primer F5 and a VP2 reverse primer R5 as shown below were designed based on the nucleotide residues at positions 1,293-1,311 and 1,382-1,394 in the plasmid pGEX-6P-1-VP2 of SEQ ID NO: 22, respectively.

```
VP2 forward primer F5
(SEQ ID NO: 58)
5′-gaattcatggaggaccgatcaacccaag-3′
      EcoRI
VP2 reverse primer R5
(SEQ ID NO: 59)
5′-ctcgagctgggagtagtgg-3′
      XhoI
```

in which the underlined nucleotides represent the recognition site of a restriction enzyme as indicated below.

With the plasmid pGEX-6P-1-VP2 as a template, a PCR product (117 bps) containing the VP2 (112-145)-encoding sequence was obtained from a PCR experiment using the VP2 forward primer F5 and the VP2 reverse primer R5 described above and the PCR reaction conditions as shown in Table 1, except that in the 30 cycles of reactions, denaturation was conducted at 95° C. for 30 sec, primer annealing was conducted at 55° C. for 30 sec, and extension was conducted at 72° C. for 1 min, followed by a 2% agarose gel electrophoresis for molecular weight verification, and recovery and purification using the QIAquick PCR Purification Kit.

A recombinant plasmid harboring said PCR product was subsequently obtained using the yT&A® Cloning Vector Kit, followed by transformation using competent E. coli cells according to the procedures as described in the General Experimental Procedures, and extraction using the QIAGEN Plasmid Mini Kit. According to the sequencing analysis conducted by Genomics Biosci & Tech Co. Ltd., the recombinant plasmid (2,845 bp), which was named pVP2 (112-145)-yT&A, was confirmed to carry the VP2 (112-145)-encoding sequence.

### B. Preparation of a DNA Hybrid Carrying a VP2 (133-138)-Encoding Sequence

In order to clone a nucleotide sequence encoding VP2 (133-138) of SEQ ID NO: 5, two DNA fragments as shown below were designed based on the nucleotide residues at positions 1,356-1,373 in the plasmid pGEX-6P-1-VP2 of SEQ ID NO: 22, respectively.

VP2 (133-138) sense fragment
(SEQ ID NO: 60)
5′-aattcatgaaacgagctaaaagaaagc-3′
VP2 (133-138) antisense fragment
(SEQ ID NO: 61)
5′-tcgagctttcttttagctcgtttcatg-3′

The two DNA fragments were subjected to a hybridization experiment using the reaction conditions as shown in Table 7, so that a DNA hybrid containing a nucleotide sequence encoding VP2 (133-138) was obtained. In addition, the DNA hybrid was formed with two sticky ends that enabled the DNA hybrid to ligate with a EcoRI/XhoI digested DNA fragment.

TABLE 7
Reaction conditions for hybridization experiment

| Contents | Volume (μL) |
|---|---|
| VP2 (133-138) sense fragment (10 μM) | 4 |
| VP2 (133-138) antisense fragment (10 μM) | 4 |
| ULTRAhyb ®-Oligo hybridization buffer (10X) | 1 |
| ddH₂O | 1 |

Operation conditions: Denaturation at 95° C. for 5 min, followed by annealing at 25° C. for 1 hour.

C. Construction of Recombinant Plasmids pcDNA3.1-VP2 (112-145)-GFP and pcDNA3.1-VP2 (133-138)-GFP

Recombinant plasmid pcDNA3.1-VP2 (112-145)-GFP (6,330 bps), which carried a nucleotide sequence encoding a VP2 (112-145)-GFP fusion protein, was obtained substantially according to the procedures as set forth in section A of Example 1 for the construction of recombinant plasmid pcDNA3.1-VP2-GFP, except that the recombinant plasmid pVP2 (112-145)-yT&A as obtained above was used in place of recombinant plasmid pVP2-yT&A.

Recombinant plasmid pcDNA3.1-VP2 (133-138)-GFP (6,246 bps), which carried a nucleotide sequence encoding a VP2 (133-138)-GFP fusion protein, was likewise obtained using the DNA hybrid as obtained above.

### D. Localization of VP2 (112-145)-GFP and VP2 (133-138)-GFP Fusion Proteins in Mammalian Cells

Recombinant plasmids pcDNA3.1-VP2 (112-145)-GFP and pcDNA3.1-VP2 (133-138)-GFP as obtained above were transfected into HeLa cells or CHO cells according to the procedures as described in the General Experimental Procedures, respectively, and the transfected cells thus obtained were subjected to fluorescence observation according to the procedures as described in the General Experimental Procedures.

### Results:

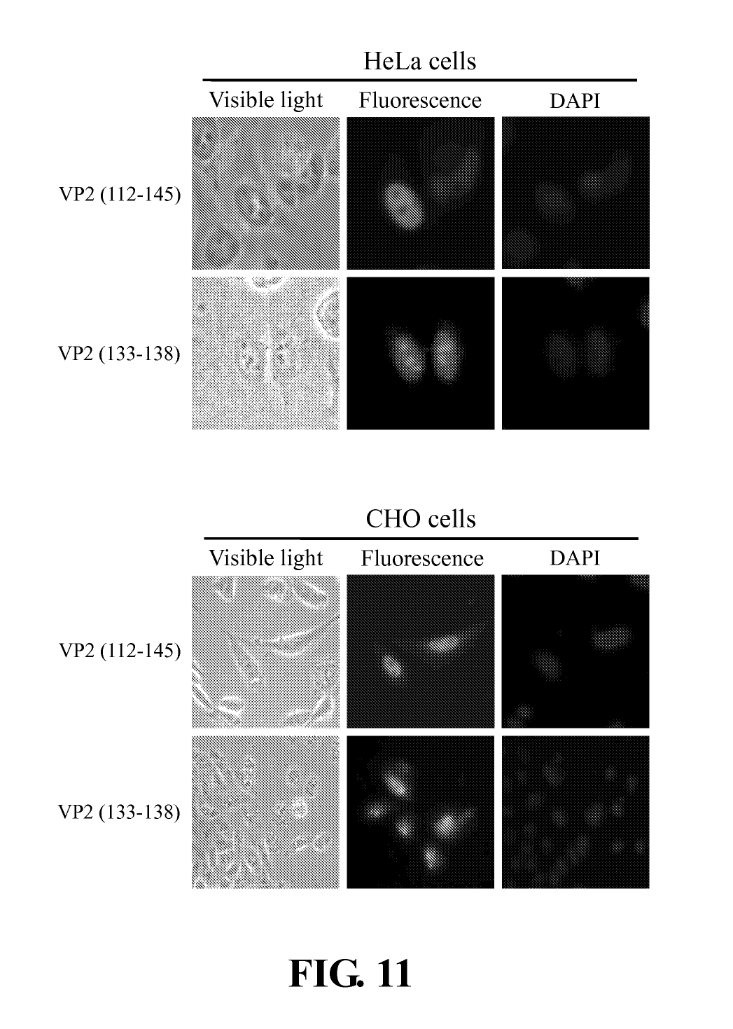

FIG. 11 shows the microscopic examination results of HeLa cells and CHO cells after transfection with recombinant plasmids pcDNA3.1-VP2 (112-145)-GFP (represented by “VP2 (112-145)”) and pcDNA3.1-VP2 (133-138)-GFP (represented by “VP2 (133-138)”), as observed by a Zeiss AxioVert 200 inverted microscope under 400× magnification. It can be seen from FIG. 11 that densely emitted green fluorescence was observed in the nucleus areas of cells transfected with either the plasmid pcDNA3.1-VP2 (112-145)-GFP or the plasmid pcDNA3.1-VP2 (133-138)-GFP. The obtained results revealed that the nuclear localization abilities of the VP2 (112-145) and VP2 (133-138) peptides might be attributed to the NLS motif of SEQ ID NO: 5 contained therein.

### Example 6

### The Influence of Point Mutations on Nuclear Localization Ability of the VP2 (112-145) Peptide

To verify the influence of point mutations on the nuclear localization ability of the VP2 (112-145) peptide constructed in Example 5, in this example, the applicants constructed four mutants of the VP2 (112-145) peptide, namely VP2 (112-145)-136-138A, VP2 (112-145)-136-138A/133A-GFP, VP2 (112-145)-136-138A/134A-GFP and VP2 (112-145)-136-138A/133A/134A.

### Experimental Procedures:

Recombinant plasmid pcDNA3.1-VP2 (112-145)-136-138A-GFP was obtained substantially according to the procedures as set forth in Example 4 for the construction of recombinant plasmid pcDNA3.1-VP2-136-138A-GFP, except for using the recombinant plasmid pcDNA3.1-VP2 (112-145)-GFP obtained in Example 5 as a template and the PCR reaction conditions shown in Table 8. Recombinant plasmid pcDNA3.1-VP2 (112-145)-136-138A-GFP carries a nucleotide sequence that encodes a mutant VP2 (112-145)-GFP fusion protein, in which the mutant VP2 (112-145) peptide contained therein has alanine substitutions at positions corresponding to amino acid residues 136-138 of the CAV VP2 protein.

Recombinant plasmids pcDNA3.1-VP2 (112-145)-136-138A/133A-GFP, pcDNA3.1-VP2 (112-145)-136-138A/134A-GFP and pcDNA3.1-VP2 (112-145)-136-138A/133A/134A-GFP were obtained substantially according to the procedures as set forth in Example 4 for the construction of recombinant plasmids pcDNA3.1-VP2-136-138A/133A-GFP, pcDNA3.1-VP2-136-138A/134A-GFP and pcDNA3.1-VP2-136-138A/133A/134A-GFP, respectively, except for using the recombinant plasmid pcDNA3.1-VP2 (112-145)-136-138A-GFP obtained above as a template and the PCR reaction conditions shown in Table 8. These three recombinant plasmids respectively carry a nucleotide sequence that encodes a mutant VP2 (112-145)-GFP fusion protein, in which the mutant VP2 (112-145) peptide contained therein is “VP2 (112-145)-136-138A/133A” for the recombinant plasmid pcDNA3.1-VP2 (112-145)-136-138A/133A-GFP, “VP2 (112-145)-136-138A/134A” for the recombinant plasmid pcDNA3.1-VP2 (112-145)-136-138A/134A-GFP, and VP2 (112-145)-136-138A/133A/134A for the recombinant plasmid pcDNA3.1-VP2 (112-145)-136-138A/133A/134A-GFP.

TABLE 8
Reaction conditions for the site-directed mutagenesis
of VP2 (112-145) encoding sequence by PCR

| Contents | Volume (μL) |
|---|---|
| A selected plasmid template (0.1 μg/μL) | 1 |
| A selected sense primer (12.5 μM) | 2 |
| A selected anti-sense primer (12.5 μM) | 2 |
| dNTPs (2.5 mM) | 4 |
| Pfu DNA polymerase buffer (10 X) | 5 |
| PfuUltra ™ II Fusion HS DNA polymerase (5 U/μL) | 1 |
| MgSO₄ (50 mM) | 2 |
| ddH₂O | 33 |

Operation conditions: Denaturation at 95° C. for 5 min, followed by 30 cycles of the following reactions: denaturation at 95° C. for 60 sec, primer annealing at 55° C. for 60 sec, and extension at 72° C. for 7 min; and finally elongation at 72° C. for 5 min.

The four recombinant plasmids thus obtained as well as recombinant plasmid pcDNA3.1-VP2 (112-145)-GFP were subsequently transfected into HeLa cells according to the procedures as described in the General Experimental Procedures, respectively, and the transfected cells thus obtained were subjected to fluorescence observation according to the procedures as described in the General Experimental Procedures.

### Results:

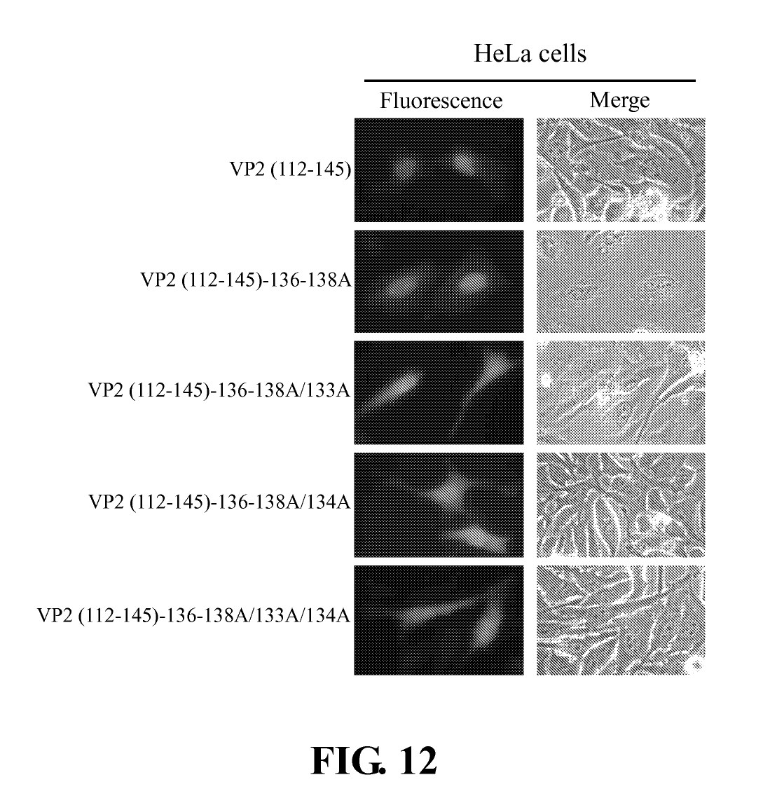

FIG. 12 shows the microscopic examination results of HeLa cells after transfection with recombinant plasmids pcDNA3.1-VP2 (112-145)-GFP (represented by the “VP2 (112-145)”), pcDNA3.1-VP2 (112-145)-136-138A-GFP (represented by the “VP2 (112-145)-136-138A”), pcDNA3.1-VP2 (112-145)-136-138A/133A-GFP (represented by “VP2 (112-145)-136-138A/133A”), pcDNA3.1-VP2 (112-145)-136-138A/134A-GFP (represented by “VP2 (112-145)-136-138A/134A”) and pcDNA3.1-VP2 (112-145)-136-138A/133A/134A-GFP (represented by “VP2 (112-145)-136-138A/133A/134A”), as observed by a Zeiss AxioVert 200 inverted microscope under 400× magnification.

Referring to FIG. 12, densely emitted green fluorescence was observed in the nucleus areas of cells transfected with plasmids pcDNA3.1-VP2 (112-145)-GFP and pcDNA3.1-VP2 (112-145)-136-138A-GFP, whereas evenly distributed green fluorescence was observed in the cytoplasm areas of cells transfected with either one of plasmids pcDNA3.1-VP2 (112-145)-136-138A/133A-GFP, pcDNA3.1-VP2 (112-145)-136-138A/134A-GFP and pcDNA3.1-VP2 (112-145)-136-138A/133A/134A-GFP. The obtained results reveal that the amino acid residues that correspond to amino acid positions 133-134 and 136-138 of the CAV VP2 protein might play an important role in the nuclear localization ability of the VP2 (112-145) peptide. This finding is consistent with that observed for the full-length CAV VP2 protein.

In view of the above Examples, it is contemplated that the VP2 NLS peptides of this invention may have a wide range of use in the delivery of effectors, such as proteins, peptides, nucleic acids, pharmaceutically active agents, chemical substances, etc., into the nucleus of a target cell.

All patents and literature references cited in the present specification as well as the references described therein, are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

We claim:

1. An isolated peptide having nuclear localization activity, wherein the isolated peptide has an amino acid sequence that:
(i) corresponds to that of a wild-type CAV VP2 protein having 216 amino acids in length, except that amino acid residues at positions 133 and/or 134 of the wild-type CAV VP2 protein are replaced to alanine, or amino acid residues at positions 136-138 of the wild-type VP2 protein are replaced to alanine, or amino acid residues at positions 150-152 of the wild-type VP2 protein are replaced to alanine, or amino acid residues at positions 136-138 and 150-152 of the wild-type VP2 protein are replaced to alanine; or
(ii) corresponds to that of a C-terminally truncated product of the wild-type CAV VP2 protein, in which amino acid residues at positions 133-138 of the wild-type CAV VP2 protein are unchanged after C-terminal truncation; or
(iii) corresponds to that of a N-terminally truncated product of the wild-type CAV VP2 protein, in which amino acid residues at positions 133-138 of the wild-type CAV VP2 protein are unchanged after N-terminal truncation; or
(iv) corresponds to that of a N-terminally and C-terminally truncated product of the wild-type CAV VP2 protein, in which amino acid residues at positions 133-138 of the wild-type CAV VP2 protein are unchanged after N-terminal and C-terminal truncations; or
(v) is represented by formula (I):
Lys-Arg-Ala-X₁—X₂—X₃—Z (I)
wherein:
X₁, X₂ and X₃ independently represent an amino acid selected from Ala, Lys and Arg; and
Z is absent or represents Leu, Leu-Asp or Leu-Asp-Tyr.

2. The isolated peptide of claim 1, wherein the wild-type CAV VP2 protein is derived from any of the following isolated strains of CAV: CAV Taiwan CIA-89 strain, CAV Australia/CAU269-7/2000 strain (UniProtKB Accession Number: Q9IZU7), CAV Germany Cuxhaven-1 strain (UniProtKB Accession Number: P69484), CAV Japan 82-2 strain (UniProtKB Accession Number: P54093), CAV USA 26p4 strain (UniProtKB Accession Number: P54092), and CAV USA CIA-1 strain (UniProtKB Accession Number: P69485).

3. The isolated peptide of claim 1, wherein the wild-type CAV VP2 protein has an amino acid sequence of SEQ ID NO: 8 or SEQ ID NO: 9 or SEQ ID NO: 10 or SEQ ID NO: 11 or SEQ ID NO: 12 or SEQ ID NO: 13.

4. The isolated peptide of claim 1, wherein the isolated peptide has an amino acid sequence selected from SEQ ID NO: 14, SEQ ID NO: 15, SEQ ID NO: 16, SEQ ID NO: 17, SEQ ID NO: 18 and SEQ ID NO: 19.

5. The isolated peptide of claim 1, wherein the C-terminally truncated product of the wild-type CAV VP2 protein has an amino acid sequence of SEQ ID NO: 20.

6. The isolated peptide of claim 1, wherein the N-terminally truncated product of the wild-type CAV VP2 protein has an amino acid sequence of SEQ ID NO: 21.

7. The isolated peptide of claim 1, wherein the N-terminally and C-terminally truncated product of the wild-type CAV VP2 protein has an amino acid sequence of SEQ ID NO: 5, SEQ ID NO: 6 or SEQ ID NO: 7.

8. The isolated peptide of claim 1, wherein the isolated peptide is chemically, enzymatically or recombinantly synthesized, or is derived from a natural source.

9. The isolated peptide of claim 1, wherein the isolated peptide is synthesized as a fusion protein.

10. The isolated peptide of claim 1, wherein the fusion protein further comprises a target protein to be transported into the nucleus of a mammalian cell.

11. A nuclear transport system comprising a target substance to be delivered into the nucleus of a mammalian cell, wherein the target substance is associated with an isolated peptide according to claim 1.

12. The nuclear transport system of claim 11, wherein the target substance is selected from the group consisting of proteins, peptides, nucleic acid molecules, pharmaceutically active agents, chemical substances, lipids, carbohydrates, and combinations thereof.

13. The nuclear transport system of claim 11, wherein the isolated peptide according to claim 1 and the target substance together form a conjugate.

14. The nuclear transport system of claim 12, wherein the target substance is a protein or peptide that forms a fusion protein with the isolated peptide.

15. The nuclear transport system of claim 9, further comprising a binding reagent that enables the nuclear transport system to enter into the mammalian cell before the target substance is transported into the nucleus of the mammalian cell.

16. A nucleic acid construct encoding a fusion protein comprising an isolated peptide according to claim 1 and a target protein to be delivered into the nucleus of a mammalian cell, wherein the nucleic acid construct comprises a first nucleic acid fragment encoding the isolated peptide, and a second nucleic acid fragment fused with the first nucleic acid fragment and encoding the target protein.

17. An expression cassette capable of expressing a fusion protein comprising an isolated peptide according to claim 1 and a target protein to be delivered into the nucleus of a mammalian cell, wherein the expression cassette comprises the nucleic acid construct of claim 16 and a promoter operably linked to the nucleic acid construct.

18. A recombinant vector carrying the expression cassette of claim 17.


SEQUENCE LISTING (61 sequences)

SEQ ID NO: 1 - protein, 7 aa, Artificial Sequence
  the SV40 large T-antigen NLS
PKKKRKV

SEQ ID NO: 2 - protein, 16 aa, Artificial Sequence
  the nucleoplasmin NLS
KRPAATKKAGQAKKKK

SEQ ID NO: 3 - DNA, 648 bp, the CAV Taiwan CIA-89 strain
atgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaaacactggtttcaagaatgtgccggacttgaggaccgatcaacccaagcctccctcgaagaagcgatcctgcgacccctccgagtacagggtaaacgagctaaaagaaagcttgattaccactactcccagccgaccccgaaccgcaagaaggtgtataagactgtaagatggcaagacgagctcgcagaccgagaggccgattttacgccttcagaagaggacggtggcaccacctcaagcgacttcgacgaagatataaatttcgacatcggaggagacagcggtatcgtagacgagcttttaggaaggcctttcacgacccccgccccggtacgtatagtg

SEQ ID NO: 4 - protein, 17 aa, Artificial Sequence
  BiNLS1 motif predicted by the WoLF PSORT software
KRKLDYHYSQPTPNRKK

SEQ ID NO: 5 - protein, 6 aa, Artificial Sequence
  NLS2 motif predicted by the NLStradamus software
KRAKRK

SEQ ID NO: 6 - protein, 9 aa, Artificial Sequence
  amino acid residues 133-141 of the CAV VP2 protein
KRAKRKLDY

SEQ ID NO: 7 - protein, 34 aa, Artificial Sequence
  amino acid residues 112-145 of the CAV VP2 protein
EDRSTQASLEEAILRPLRVQGKRAKRKLDYHYSQ

SEQ ID NO: 8 - protein, 216 aa, the CAV Taiwan CIA-89 strain
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAKRKLDYHYSQPTPNRKKVYKTVRWQDELADREADFTPSEEDGGTTSSDFDEDINFDIGGDSGIVDELLGRPFTTPAPVRIV

SEQ ID NO: 9 - protein, 216 aa, the CAV Australia/CAU269-7/2000 strain
MHGNGGQPAAGGSESALSREGQPGPSGAAQGQVISNERSPRRYSTRTINGVQATNKFTAVGNPSLQRDPDWYRWNYSHSIAVWLRECSRSHAKICNCGQFRKHWFQECAGLEDRSTQASLEEAILRPLRVQGKRAKRKLDYHYSQPTPNRKKVYKTVRWQDELADREADFTPSEEDGGTTSSDFDEDINFDIGGDSGIVDELLGRPFTTPAPVRIV

SEQ ID NO: 10 - protein, 216 aa, the CAV Germany Cuxhaven-1 strain; identical to SEQ ID NO: 8

SEQ ID NO: 11 - protein, 216 aa, the CAV Japan 82-2 strain
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAKRKLDYHYSQPTPNRKKVYKTVRWKDELADREADFTPSEEDGGTTSSDFDEDINFDIGGDSGIVDELLGRPFTTPAPVRIV

SEQ ID NO: 12 - protein, 216 aa, the CAV USA 26p4 strain
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAKRKLDYHYSQPTPNRKKVYKTVRWQDELADREADFTPSEEDGGTTSSDFDGDINFDIGGDSGIVDELLGRPFTTPAPVRIV

SEQ ID NO: 13 - protein, 216 aa, the CAV USA CIA-1 strain; identical to SEQ ID NO: 8

SEQ ID NO: 14 - protein, 216 aa, Artificial Sequence
  the amino acid sequence of the mutant VP2 protein of the CAV Taiwan CIA-89 strain (VP2-150-152A)
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAKRKLDYHYSQPTPNAAAVYKTVRWQDELADREADFTPSEEDGGTTSSDFDEDINFDIGGDSGIVDELLGRPFTTPAPVRIV

SEQ ID NO: 15 - protein, 216 aa, Artificial Sequence
  the amino acid sequence of the mutant VP2 protein of the CAV Taiwan CIA-89 strain (VP2-136-138A)
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAAAALDYHYSQPTPNRKKVYKTVRWQDELADREADFTPSEEDGGTTSSDFDEDINFDIGGDSGIVDELLGRPFTTPAPVRIV

SEQ ID NO: 16 - protein, 216 aa, Artificial Sequence
  the amino acid sequence of the mutant VP2 protein of the CAV Taiwan CIA-89 strain (VP2-136-138A/150-152A)
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAAAALDYHYSQPTPNAAAVYKTVRWQDELADREADFTPSEEDGGTTSSDFDEDINFDIGGDSGIVDELLGRPFTTPAPVRIV

SEQ ID NO: 17 - protein, 216 aa, Artificial Sequence
  the amino acid sequence of the mutant VP2 protein of the CAV Taiwan CIA-89 strain (VP2-133A)
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGARAKRKLDYHYSQPTPNRKKVYKTVRWQDELADREADFTPSEEDGGTTSSDFDEDINFDIGGDSGIVDELLGRPFTTPAPVRIV

SEQ ID NO: 18 - protein, 216 aa, Artificial Sequence
  the amino acid sequence of the mutant VP2 protein of the CAV Taiwan CIA-89 strain (VP2-134A)
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKAAKRKLDYHYSQPTPNRKKVYKTVRWQDELADREADFTPSEEDGGTTSSDFDEDINFDIGGDSGIVDELLGRPFTTPAPVRIV

SEQ ID NO: 19 - protein, 216 aa, Artificial Sequence
  the amino acid sequence of the mutant VP2 protein of the CAV Taiwan CIA-89 strain (VP2-133A/134A)
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGAAAKRKLDYHYSQPTPNRKKVYKTVRWQDELADREADFTPSEEDGGTTSSDFDEDINFDIGGDSGIVDELLGRPFTTPAPVRIV

SEQ ID NO: 20 - protein, 145 aa, Artificial Sequence
  the amino acid sequence of the C-terminal truncated VP2 protein of the CAV Taiwan CIA-89 strain (VP2-145dC)
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAKRKLDYHYSQ

SEQ ID NO: 21 - protein, 105 aa, Artificial Sequence
  the amino acid sequence of the N-terminal truncated VP2 protein of the CAV Taiwan CIA-89 strain (VP2-111dN)
EDRSTQASLEEAILRPLRVQGKRAKRKLDYHYSQPTPNRKKVYKTVRWQDELADREADFTPSEEDGGTTSSDFDEDINFDIGGDSGIVDELLGRPFTTPAPVRIV

SEQ ID NO: 22 - DNA, 5626 bp, Artificial Sequence
  plasmid pGEX-6P-1-VP2
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattcatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaaacactggtttcaagaatgtgccggacttgaggaccgatcaacccaagcctccctcgaagaagcgatcctgcgacccctccgagtacagggtaaacgagctaaaagaaagcttgattaccactactcccagccgaccccgaaccgcaagaaggtgtataagactgtaagatggcaagacgagctcgcagaccgagaggccgattttacgccttcagaagaggacggtggcaccacctcaagcgacttcgacgaagatataaatttcgacatcggaggagacagcggtatcgtagacgagcttttaggaaggcctttcacgacccccgccccggtacgtatagtgtgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 23 - DNA, 6252 bp, Artificial Sequence
  plasmid pcDNA3.1-GFP
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattctgcagatatccagcacagtggcggccgctcgagcccgggatggtagatctgactagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctagccaccaccaccaccaccacgtgtgaattccgcggttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 24 - DNA, 26 bp, Artificial Sequence
  VP2 forward primer F1
tggaattcatgcacgggaacggcgga

SEQ ID NO: 25 - DNA, 23 bp, Artificial Sequence
  VP2 reverse primer R1
tcctcgagcactatacgtaccgg

SEQ ID NO: 26 - DNA, 24 bp, Artificial Sequence
  VP2 reverse primer R2
tcctcgagtgatcggtcctcaagt

SEQ ID NO: 27 - DNA, 23 bp, Artificial Sequence
  VP2 reverse primer R3
tcctcgagaccctgtactcggag

SEQ ID NO: 28 - DNA, 26 bp, Artificial Sequence
  VP2 reverse primer R4
tcctcgagctgggagtagtggtaatc

SEQ ID NO: 29 - DNA, 26 bp, Artificial Sequence
  VP2 forward primer F2
tggaattcatggaggaccgatcaacc

SEQ ID NO: 30 - DNA, 26 bp, Artificial Sequence
  VP2 forward primer F3
aggaattcatgcactactcccagccg

SEQ ID NO: 31 - DNA, 26 bp, Artificial Sequence
  VP2 forward primer F4
aggaattcatggacgagctcgcagac

SEQ ID NO: 32 - DNA, 27 bp, Artificial Sequence
  Sense primer MF1
aaacgagctgctgctgctcttgattac

SEQ ID NO: 33 - DNA, 27 bp, Artificial Sequence
  Anti-sense primer MR1
gtaatcaagagcagcagcagctcgttt

SEQ ID NO: 34 - DNA, 39 bp, Artificial Sequence
  Sense primer MF2
accccgaacgcagcagcagtgtataagactgtaagatgg

SEQ ID NO: 35 - DNA, 39 bp, Artificial Sequence
  Anti-sense primer MR2
ccatcttacagtcttatacactgctgctgcgttcggggt

SEQ ID NO: 36 - DNA, 27 bp, Artificial Sequence
  Sense primer MF3
gtacagggtaaagctgctgctgctgct

SEQ ID NO: 37 - DNA, 27 bp, Artificial Sequence
  Anti-sense primer MR3
agcagcagcagcagctttaccctgtac

SEQ ID NO: 38 - DNA, 27 bp, Artificial Sequence
  Sense primer MF4
gtacagggtgctcgagctgctgctgct

SEQ ID NO: 39 - DNA, 27 bp, Artificial Sequence
  Anti-sense primer MR4
agcagcagcagctcgagcaccctgtac

SEQ ID NO: 40 - DNA, 27 bp, Artificial Sequence
  Sense primer MF5
gtacagggtgctgctgctgctgctgct

SEQ ID NO: 41 - DNA, 27 bp, Artificial Sequence
  Anti-sense primer MR5
agcagcagcagcagcagcaccctgtac

SEQ ID NO: 42 - DNA, 28 bp, Artificial Sequence
  Sense primer MF6
gtacagggtgctcgagctaaaagaaagc

SEQ ID NO: 43 - DNA, 28 bp, Artificial Sequence
  Anti-sense primer MR6
gctttcttttagctcgagcaccctgtac

SEQ ID NO: 44 - DNA, 28 bp, Artificial Sequence
  Sense primer MF7
gtacagggtaaagctgctaaaagaaagc

SEQ ID NO: 45 - DNA, 28 bp, Artificial Sequence
  Anti-sense primer MR7
gctttcttttagcagctttaccctgtac

SEQ ID NO: 46 - DNA, 28 bp, Artificial Sequence
  Sense primer MF8
gtacagggtgctgctgctaaaagaaagc

SEQ ID NO: 47 - DNA, 28 bp, Artificial Sequence
  Anti-sense primer MR8
gctttcttttagcagcagcaccctgtac

SEQ ID NO: 48 - protein, 20 aa, Artificial Sequence
  amino acid residues 133-152 of the mutant VP2 protein (VP2-150-152A)
KRAKRKLDYHYSQPTPNAAA

SEQ ID NO: 49 - protein, 20 aa, Artificial Sequence
  amino acid residues 133-152 of the mutant VP2 protein (VP2-136-138A)
KRAAAALDYHYSQPTPNRKK

SEQ ID NO: 50 - protein, 20 aa, Artificial Sequence
  amino acid residues 133-152 of the mutant VP2 protein (VP2-136-138A/150-152A)
KRAAAALDYHYSQPTPNAAA

SEQ ID NO: 51 - protein, 20 aa, Artificial Sequence
  amino acid residues 133-152 of the mutant VP2 protein (VP2-136-138A/133A)
ARAAAALDYHYSQPTPNRKK

SEQ ID NO: 52 - protein, 20 aa, Artificial Sequence
  amino acid residues 133-152 of the mutant VP2 protein (VP2-136-138A/134A)
KAAAAALDYHYSQPTPNRKK

SEQ ID NO: 53 - protein, 20 aa, Artificial Sequence
  amino acid residues 133-152 of the mutant VP2 protein (VP2-136-138A/133A/134A)
AAAAAALDYHYSQPTPNRKK

SEQ ID NO: 54 - protein, 20 aa, Artificial Sequence
  amino acid residues 133-152 of the mutant VP2 protein (VP2-133A)
ARAKRKLDYHYSQPTPNRKK

SEQ ID NO: 55 - protein, 20 aa, Artificial Sequence
  amino acid residues 133-152 of the mutant VP2 protein (VP2-134A)
KAAKRKLDYHYSQPTPNRKK

SEQ ID NO: 56 - protein, 20 aa, Artificial Sequence
  amino acid residues 133-152 of the mutant VP2 protein (VP2-133A/134A)
AAAKRKLDYHYSQPTPNRKK

SEQ ID NO: 57 - protein, 6 aa, Artificial Sequence
  Ala mutant form of the NLS2 motif predicted by the NLStradamus software
KRAAAA

SEQ ID NO: 58 - DNA, 28 bp, Artificial Sequence
  VP2 forward primer F5
gaattcatggaggaccgatcaacccaag

SEQ ID NO: 59 - DNA, 19 bp, Artificial Sequence
  VP2 reverse primer R5
ctcgagctgggagtagtgg

SEQ ID NO: 60 - DNA, 27 bp, Artificial Sequence
  VP2 (133-138) sense fragment
aattcatgaaacgagctaaaagaaagc

SEQ ID NO: 61 - DNA, 27 bp, Artificial Sequence
  VP2 (133-138) antisense fragment
tcgagctttcttttagctcgtttcatg